(19) United States
(12) Patent Application Publication
(10) Pub. No.: US 2005/0268909 A1
(43) Pub. Date: Dec. 8, 2005

(54) Medicament dispenser

(76) Inventors: Stanley George Bonney, Ware (GB); Michael Birsha Davies, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/523,121

(22) PCT Filed: Jul. 23, 2003
(86) PCT No.: PCT/EP03/08151

§ 371 Date: Jan. 24, 2005

(30) Foreign Application Priority Data
Jul. 25, 2002 (GB) 0217196.5

(51) Int. Cl. A61M 15/00

(52) U.S. Cl. 128/203.15; 128/203.12

(57) ABSTRACT

A medicament dispenser device for use in the delivery of a multi-component combination medicament product. The device comprises a first medicament container containing plural co-formulation compatible medicament components; first release means for releasing the contents of the first medicament container for delivery thereof; at least one further medicament container, each containing at least one co-formulation incompatible medicament component; and at least one further release means for releasing the contents of each at least one further medicament container for delivery thereof. The at least one co-formulation incompatible medicament component is kept separate from the plural co-formulation compatible components until the point of release thereof for delivery in combination.

### TECHNICAL FIELD

The present invention relates to a medicament dispenser for dispensing ‘multi-active’ medicament combination products. The invention particularly relates to an inhaler device for use in dispensing medicament in combination product form.

### BACKGROUND TO THE INVENTION

The use of inhalation devices in the administration of medicaments, for example in bronchodilation therapy is well known. Such devices generally comprise a body or housing within which a medicament carrier is located. Known inhalation devices include those in which the medicament carrier is a blister strip containing a number of discrete doses of powdered medicament. Such devices usually contain a mechanism of accessing these doses, usually comprising either piercing means or means to peel a lid sheet away from a base sheet. The powdered medicament can hen be accessed and inhaled. Other known devices include those in which the medicament is delivered in aerosol form, including the well-known metered dose inhaler (MDI) delivery devices. Liquid-based inhaler devices are also known.

Therapies involving combinations of different and complementary active medicaments are known. These can be administered either as distinct combination (i.e. plural-active) medicament products, which comprise a defined mixture of each component medicament, or as groups of single active medicament products, which are designed to be taken in combination or sequentially. Whilst combination products offer added convenience for the patient, certain medicament actives are difficult to formulate as distinct combination products. For example, the co-actives may interact chemically with each other in an undesirable way when formulated together.

The problem of undesirable physical or chemical interaction between different medicament constituents can be especially complex and difficult to address for combination products combining three or more different medicaments in a single-formulated product (a ‘multi-active combination product’). In particular, one or more of the medicament components may be non-complementary with one or more of the other medicament components. This complexity can potentially either restrict development choice, make formulation development more complex or lead to compromise in pharmaceutical performance, when developing any particular multi-active combination product.

Whilst the above problem may be addressed by separate formulation of the various components and sequential delivery thereof by separate and different types of inhaler, this is inconvenient for the patient and can lead to confusion in situations where for example, the different inhalers are of different types or contain different numbers of doses of medicament.

The Applicants therefore now provide, in solution to the above problem, a medicament dispenser device (and packs suitable for use therewith) that enables the separate (i.e. in isolated fashion) containment of a first product comprising plural compatible medicament components, and of one or more incompatible medicament components. Here the term ‘compatible’ is used to mean compatible in the sense of being amenable to co-formulation with each other.

The device enables the delivery of all of the medicament components together as a combined product for administration to the patent. Suitably, the combined dose is deliverable in response to a minimum number of patient actions. In particular, it is desirable that each active component of the combined dose is delivered to the patient in a single, combined dose in response to a single patient dosing action. For example, it is desirable that a combined product for inhalation be delivered in response to a single patient actuation of an inhaler, even where the incompatible active components of that combined product are separately stored within the inhaler device.

The Applicants have also observed that particular medicaments and combinations thereof can be more suited for formulation and delivery by particular types of inhaler device. For example, one particular medicament (or combination) may be more suitable for delivery by an MDI device, whereas another may be more suitable for delivery by a DPI device. That suitability may for example, be driven by ease of formulation of the medicament for that particular inhaler device or by the delivery and pharmaceutical performance characteristics obtainable when the particular inhaler device is employed.

The Applicants have also now realised that this preferential suitability can potentially either restrict development choice, or lead to compromise in pharmaceutical performance, when developing an inhaler device for any particular multi-active combination product. The problem is encountered where one or more component medicaments of the combination product is suited to delivery by one particular type of inhaler device, whereas one or more of the other component medicaments of that combination product is suited to delivery by a different type of inhaler device.

The Applicants therefore now provide, in solution to the above problem, a single inhaler device, which comprises in combination different inhaler types (e.g. combined DPI and MDI; MDI and LSI; or DPI and LSI). The device enables convenient, combined delivery of the components of a combination medicament product to a patient. Suitably, the delivery of the medicament combination occurs on a simultaneous basis and is responsive to a minimum number of patient actions (e.g. single patient actuation or inhalation step).

The Applicants have also realised that using a dispenser device of the type herein provided can potentially reduce the complexity, timescale and cost of the development process for a particular ‘multi-active’ combination product because it enables the optimum (e.g. from a development simplicity) delivery vehicle to be selected for each particular medicament component of the combination. The additional development complexity, which is often associated with formulating combination products is thereby effectively avoided.

### SUMMARY OF THE INVENTION

According to one aspect of the invention there is provided a medicament dispenser device for use in the delivery of a multi-component combination medicament product, the device comprising
a first medicament container containing plural co-formulation compatible medicament components;
first release means for releasing the contents of the first medicament container for delivery thereof;
at least one further medicament container, each containing at least one co-formulation incompatible medicament component; and
at least one further release means for releasing the contents of each at least one further medicament container for delivery thereof,
wherein the at least one co-formulation incompatible medicament component is kept separate from the plural co-formulation compatible components until the point of release thereof for delivery in combination.

The term ‘co-formulation compatible’ is used to mean compatible in the sense of being amenable to co-formulation, perhaps even displaying synergetic co-formulation characteristics. The term ‘co-formulation incompatible’ is used to mean the reverse, that is to say for whatever reason including chemical or physical incompatibility or simply lack of synergetic characteristics or benefits, the medicament components are either non-amenable to co-formulation or for whatever reason, including for development simplicity, preferably not co-formulated.

In combination, the co-formulation compatible medicament components and at least co-formulation incompatible medicament component comprise a defined combination product. That is to say, that when combined together the distinct active medicament doses released by actuation of the device form a dose of a ‘multi-active’ medicament treatment.

In aspects, the plural co-formulation compatible medicament components and the at least one co-formulation incompatible medicament component may be arranged for simultaneous or sequential release from the one or more medicament containers, although in general where components are released sequentially the time delay between release of each particular medicament component is short (e.g. milliseconds) to ensure that a combination product is provided for delivery to the patient.

On actuation, the dispenser device is designed to deliver a dose portion of the plural co-formulation compatible medicament components and a dose portion of each at least one co-formulation incompatible medicament. The term ‘dose portion’ is employed because in the context of the invention the distinct ‘portions’ are brought together on delivery to form a combination (i.e. multi-active) product dose.

In one particular aspect, the medicament dispenser herein comprises a first medicament container containing two co-formulation compatible medicament components and one further medicament container containing one co-formulation incompatible medicament component (i.e. a ‘2+1’ embodiment).

In another particular aspect, the medicament dispenser herein comprises a first medicament container containing two co-formulation compatible medicament components and one further medicament container containing two co-formulation incompatible medicament components (i.e. a ‘2+2’ embodiment). In this embodiment, it will be appreciated that within each set of two medicaments, the components thereof are compatible, even though they are incompatible with the components of the other set of two.

In a further particular aspect, the medicament dispenser herein comprises a first medicament container containing two-co-formulation compatible medicament components a second medicament container containing one co-formulation incompatible medicament component and a third medicament container containing one co-formulation incompatible medicament component (i.e. a ‘2+1+1’ embodiment).

The first and at least one further medicament containers may be of a similar-type or in aspects, be of a different type. This enables additional flexibility in that one container may for example, accommodate a product in dry powder form whereas the other container accommodates product in liquid, solution or aerosol form.

In one aspect, the first medicament container and the at least one further medicament container are of a type adapted to be used with a medicament dispenser selected from the group consisting of a reservoir dry powder inhaler (RDPI), a multi-dose dry powder inhaler (MDPI), a unit dose dry powder inhaler (UDPI), a metered dose inhaler (MDI) and a liquid spray inhaler (LSI). The first medicament dispenser and at least one further remain different in type.

In one aspect, the first medicament dispenser is a reservoir dry powder inhaler (RDPI), and the at least one further medicament dispenser is of a type selected from the group consisting of a multi-dose dry powder inhaler (MDPI), a metered dose inhaler (MDI) and a liquid spray inhaler (LSI).

In another aspect, the first medicament dispenser is a multi-dose dry powder inhaler (MDPI), and the at least one further medicament dispenser is of a type selected from the group consisting of a reservoir dry powder inhaler (RDPI), a metered dose inhaler (MDI) and a liquid spray inhaler (LSI).

In a further aspect, the first medicament dispenser is a metered dose inhaler (MDI), and the at least one further medicament dispenser is of a type selected from the group consisting of a reservoir dry powder inhaler (RDPI), a multi-dose dry powder inhaler (MDPI) and a liquid spray inhaler (LSI).

In a further aspect, the first medicament dispenser is a liquid spray inhaler (LSI), and the at least one further medicament dispenser is of a type selected from the group consisting of a reservoir dry powder inhaler (RDPI), a multi-dose dry powder inhaler (MDPI) and a metered dose inhaler (MDI).

By reservoir dry powder inhaler (RDPI) it is meant an inhaler having a reservoir form container pack suitable for containing multiple (un-metered doses) of medicament product in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By unit dose dry powder inhaler (UDPI) it is meant an inhaler suitable for dispensing medicament in dry powder form, wherein the medicament is comprised within a unit dose container pack containing a single dose (or part thereof of medicament product. In a preferred aspect, the carrier has a capsule-based pack form.

By multi-dose dry powder inhaler (MDPI) it is meant an inhaler suitable for dispensing medicament in dry powder form, wherein the medicament is comprised within a multi-dose container pack containing (or otherwise carrying) multiple, define doses (or parts thereof of medicament product. In a preferred aspect, the carrier has a blister pack form, but it could also, for example, comprise a capsule-based pack form or a carrier onto which medicament has been applied by any suitable process including printing, painting and vacuum occlusion.

In one aspect, the multi-dose pack is a blister pack comprising multiple blisters for containment of medicament product in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 in the name of Glaxo Group Ltd describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (I) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

By liquid spray inhaler (LSI) it is meant a medicament dispenser suitable for dispensing medicament in spray form, wherein the medicament is typically formulated in liquid or solution form and comprised in a liquid container. The container is typically provided with a means of metering to a spray generator, which imparts energy to the liquid or solution, thereby generating a spray for inhalation by the patient. The spray generator, in aspects, comprises a vibrating element (e.g. a mesh) that provides vibrational energy to the formulation, thereby resulting in its aerosolisation. In other aspects, the spray generator comprises a pump mechanism, which either delivers the medicament directly to the patient (as a liquid spray) or which delivers the medicament to an intermediate position at which further energy is supplied thereto to further propel, aerosolise or otherwise direct the medicament dose to the patient.

The medicament dispenser device herein has unitary form, and typically has a housing shaped to receive, and enable the release of medicament product from the first and at least one further medicament containers.

In one aspect, the housing integrally comprises a release means for releasing medicament from at least one, preferably all of the medicament dispensers. Suitably, the release means for each medicament container is coupled, thereby enabling simultaneous delivery of medicament from each dispenser in response to a single patient actuation step.

In another aspect, the housing is shaped to receive the medicament containers, each of which is provided with respective release means. In this case, the release means have typically been adapted for receipt by the housing. The medicament dispenser and release means therefor are in one aspect, supplied as independently operable ‘cassette refills’ for the unitary device.

In one aspect, the device is provided with mixing means for ensuring mixing of the delivered medicaments prior to their inhalation by the patient as a ‘mixed’ multi-active combination product.

Suitably, the mixing means comprises a mixing chamber including inlets for receiving medicament form each medicament container and an outlet for delivery of ‘mixed’ medicament product to the patient for inhalation (e.g. through a mouthpiece which communicates with the mixing chamber). The ergonomics of the mixing chamber will be arranged to ensure effective mixing of the separate medicament feeds. In aspects, baffles, propellers, venturi and other features for controlling mixing dynamics are provided. The mixing chamber may also be provided with energisation means for energising the mixing process, or alternatively features may be provided to harness the energy provided by a patient's inward breath to enhance the mixing process.

The dispenser device may be provided with means for varying the amount of medicament product released from each medicament container. Customized delivery of combination medicament product may therefore be achieved through varying the relative ratios of each individual medicament product delivered as well as by varying the absolute amount of medicament product delivered. Variable timing mechanisms are envisaged for achieving such customisation.

In one aspect, the medicament dispenser herein includes a timing control system for controlling the time of release of contents from the first and at least one further medicament container. The timing control system generally communicates with an electronic control system with which it may in aspects, form an integral part.

The timing control system is suitably arranged to vary the relative time of release of each medicament component from its respective medicament container. Each medicament component may therefore be arranged for simultaneous or sequential release, although in general where components are released sequentially the time delay between releases of each separate medicament component is short (e.g. milliseconds) to ensure that a combined product is provided for administration to the patient.

In a further aspect, by varying the time of release, the ratio of quantity of each medicament component released can also be varied, thereby enabling the provision and delivery of ‘tailored’ combined products.

Delivery of the combination product (e.g. after mixing) to the patient is preferably through a single outlet. The outlet is typically positioned to be in communication with the distinct medicament dose portions delivered. The outlet may have any suitable form. In one aspect, it has the form of a mouthpiece and in another it has the form of a nozzle for insertion into the nasal cavity of a patient.

The outlet is preferably a single outlet, which communicates with the distinct medicament dose portions delivered via a common air channelling means (e.g. formed as an air-pipe or common manifold). The patient may therefore breathe in through a single outlet, and that breath be transferred through the common channelling means to (all of) the released medicament dose portions, thereby enabling their inhalation as a multi-active combined product.

In addition to, or as an alternative to, any separate mixing chamber, the outlet and/or channelling means may be shaped to encourage mixing of medicament as a result of the air flow created by inhalation by the patient. For example, baffles or other mechanical aids to mixing may be incorporated. Venturi channelling of the air flow is also envisaged in embodiments. Helical form channels are envisaged.

Any or all mechanical components of the device may be driven by either an electronic or mechanical drive system or combination thereof.

Suitably electronic drive means typically comprise a motor, preferably an electrically-powered motor. The motor may provide linear or rotary drive, but in general, rotary motors are most suitable. The motor may for example, comprise a DC electric motor, a piezoelectric (PZ) motor, an ultrasonic motor, a solenoid motor or a linear motor. Preferably, the electronic drive system comprises a DC motor, a PZ motor or an ultrasonic motor.

The use of ultrasonic motors is particularly preferred since they offer advantages over conventional motors in terms of weight, size, noise, cost and torque generated.

Ultrasonic motors are well known in the art and are commercially available (e.g. BMSTU Technological Cooperation Centre Ltd, Moscow, Russia; Shinsei Corporation, Tokyo, Japan).

Ultrasonic motors do not use coils or magnets but comprise a piezoelectric ceramic stator which drives a coupled rotor. The stator generates ultrasonic vibrations which in turn causes rotation of the rotor. While regular DC motors are characterised by high speed and low torque, requiring reduction gearing to increase torque, ultrasonic motors attain low speed and high torque, thus eliminating the need for reduction gearing. Furthermore, these motors are lightweight and compact, lacking coils and magnets, and are noiseless as the ultrasonic frequencies used are not audible to the human ear.

Suitably, the device further comprises actuating means for actuating said electronic drive system. Said actuating means may take the form of a switch, push-button, or lever.

Suitably, the device additionally comprises an electronic data management system. The electronic data management system has input/output capability and comprises a memory for storage of data; a microprocessor for performing operations on said data; and a transmitter for transmitting a signal relating to the data or the outcome of an operation on the data.

Suitably, the electronic data management system is arranged to be responsive to or activated by the voice of a user. Thus, for example the system may be switched on or off in response to a voice command.

The electronic data management system may be integral with the body. Alternatively, the electronic data management system forms part of a base unit which is reversibly associable with the body.

Suitably, the device additionally comprises a data input system for user input of data to the electronic data management system. Preferably, the data input system comprises a man machine interface (MMI) preferably selected from a keypad, voice recognition interface, graphical user interface (GUI) or biometrics interface.

Energy may be conserved by a variety of means to enable the device to operate for longer on a given source of energy, such as a battery. Energy conservation or saving methods have additional advantages in terms of reducing the size requirements of the power source (e.g. battery) and thus the weight and portability of the medicament dispenser.

A variety of energy saving methods is available which generally involve reducing power consumption. One such method is to use a clock or timer circuit to switch the power on and off at regular or predetermined intervals. In another method the system can selectively switch on/off specific electronic devices, such as visual display units or sensors, in order to power these devices only when they are required to perform a particular sequence of events. Thus different electronic devices may be switched on and off at varying intervals and for varying periods under control of the system. The power sequencing system may also respond to a sensor, such as a motion or breath sensor, which is activated on use of the device.

Low power or “micropower” components should be used within the electronics where possible and if a high power device is required for a particular function this should be put into a low power standby mode or switched off when not required. Similar considerations apply in the selection of transducers. Operation at low voltage is desirable since power dissipation generally increases with voltage.

For low power digital applications complementary metal oxide semi-conductor (CMOS) devices are generally preferred and these may be specially selected by screening for low quiescent currents. Clock speeds of processors and other logic circuits should be reduced to the minimum required for computational throughput as power consumption increases with frequency. Supply voltages should also be kept at minimal values consistent with reliable operation because power dissipation in charging internal capacitance's during switching is proportional to the square of the voltage. Where possible, supply voltages should be approximately the same throughout the circuit to prevent current flowing through input protection circuits. Logic inputs should not be left floating and circuits should be arranged so that power consumption is minimised in the most usual logic output state. Slow logic transitions are undesirable because they can result in relatively large class-A currents flowing. Resistors may be incorporated in the power supply to individual devices in order to minimise current in the event of failure.

In some control applications, devices that switch between on and off states are preferred to those that allow analog (e.g. linear) control because less power is dissipated in low resistance on states and low current off states. Where linear components are used (e.g. certain types of voltage regulators) then types with low quiescent currents should be selected. In some circuit configurations it is preferable to use appropriate reactive components (i.e. inductors and capacitors) to reduce power dissipation in resistive components.

Suitably, the system additionally comprises a visual display unit for display of data from the electronic data management system to the user. The display may for example, comprise a screen such as an LED or LCD screen. More preferably the visual display unit is associable with the body of the medicament dispenser.

Suitably, the device additionally comprises a datalink for linking to a local data store to enable communication of data between the local data store and the electronic data management system. The datastore may also comprise data management, data analysis and data communication capability.

The datastore may itself form part of a portable device (e.g. a handheld device) or it may be sized and shaped to be accommodated within the patient's home. The datastore may also comprise a physical storage area for storage of replacement cassettes. The datastore may further comprise a system for refilling medicament from a reservoir of medicament product stored therewithin. The datastore may further comprise an electrical recharging system for recharging any electrical energy store on the medicament dispenser, particularly a battery recharging system.

The datalink may for example enable linking with a docking station, a personal computer, a network computer system or a set-top box by any suitable method including a hard-wired link, an infrared link or any other suitable wireless communications link.

In one aspect, the device includes an electronic dose reminder system. This may be configured to have any suitable form and may be powered by a mains, stored (e.g. battery) or self-regenerating (e.g. solar) energy power source.

The electronic dose reminder system comprises an electronic timer for timing an elapsed time period corresponding to the time since the last actuation of the device; a dose interval memory for storing data relating to a prescribed dose interval time period; and a patient alerter for alerting a user. The alerter activates when the elapsed time period exceeds the prescribed dose interval time period.

The electronic timer progressively times the period since the last actuation of the device (the ‘elapsed time period’). The timer can have any suitable electronic form. The significance of the ‘elapsed time period’ is that in use, it typically corresponds to the time elapsed since the previous dose delivery event.

The timer may be configured to include an automatic re-zeroing feature such that on subsequent actuation of the device the timer count starts again from zero.

The dose interval memory stores data relating to a prescribed dose interval time period. By way of examples, if the medicament is to be taken twice a day at a regular interval, the prescribed dose interval may be set as twelve hours, or for a once daily treatment the value may be set at twenty four hours. In aspects, the system may be configured to allow for ready readjustment of the prescribed dose interval time period, or it may be configured in secure fashion such that any readjustment may be made only by a designated prescriber (e.g. a medical professional or pharmacist). Password and/or other security means may be employed. The prescribed dose interval may be configured to be variable over a particular course of treatment, or alternatively it may be fixed at a set dose interval over the full course of treatment.

The patient alerter is designed to communicate an alert to the user. The alerter activates only when the holding time period exceeds the prescribed dose interval time period. By way of an example, for a once daily treatment with a prescribed dose interval of twenty four hours, the alerter would activate only when the holding time period, as timed by the electronic timer, exceeds twenty four hours since at this point another dose is due to be taken. It may thus, be appreciated that the alerter acts functionally as a reminder to the patient that a dose is due to be taken.

The alerter may in aspects, comprise a visual device, such as a liquid crystal display (LCD) or an array of light-emitting diodes (LEDs), connected to a battery-driven timing device of any convenient kind known to those skilled in the art. The visual device may be configured to display information such as the actual time or the elapsed time from the taking of a previous dosage and may have superimposed thereon additional messages, such as a textual instruction to take a dose of the medicament. Alternatively, the instruction to take the medicament may be conveyed merely by displaying a warning colour or by causing the display to flash or in any other way.

In a further alternative arrangement, no specific time or elapsed time information is displayed, but the alerter merely provides a warning signal that indicates the necessary action to the user.

Depending upon the lifestyle of the user, additional or alternative warnings may be of greater assistance than purely visual warnings. Accordingly, the invention envisages that the alerter may provide audible and/or tactile warnings, such as vibration, instead of (or in addition to) visual warnings.

The alerter may provide a single, one-off alert. More preferably, the alerter is configured to provide the alert over a set period of time (the ‘alerting time period’ or ‘alerting window’). In one aspect, the alerting time period is calculated as a function of (e.g. fraction of) the dose interval time period. For example, for a twice daily treatment with a dose interval time period of twelve hours, the alerting time period may be set as half that period (i.e. six hours). In this case, the alert is then provided for the six hours immediately following the activation of the alert.

The system is typically configured such that the alerting signal cuts off when the user removes the medicament delivery device from the holder to enable dosing of medicament therefrom. The system is then reset. Other manual cutoffs/overrides may also be included.

In a subtle aspect of the present invention, it may be appreciated that the relevant timeframe for detecting, timing and alerting are determined by user action in relation to the system, and in particular by user action. The dose reminder capability is therefore independent of any particular defined external time zone (e.g. the local time zone relative to Greenwich Mean Time, as defined by the twenty four hour clock) because the user action defines its own ‘reminder timeframe’. This provides advantages over other known reminder systems, which are reliant on user reference to defined external time frames. The advantage is particularly great for the international traveller since complex calculations involving different local time zones are avoided.

It will be appreciated from the above description that the various components of the electronic dose reminder system interrelate with each other to provide the required functionality. The system may be configured in any suitable fashion using known electronic components and circuitry methods.

Suitably, the device additionally comprises an actuation detector for detecting actuation of any one of the medicament dispensers thereof wherein said actuation detector transmits actuation data to the electronic data management system.

The device may additionally comprise a safety mechanism to prevent unintended multiple actuations of the component medicament dispensers. The patient is thereby, for example, protected from inadvertently receiving multiple doses of medicament in a situation where they take a number of short rapid breaths. More preferably, the safety mechanism imposes a time delay between successive actuations of the release means. The time delay is typically of the order of from three to thirty seconds.

Suitably, the device additionally comprises a release detector for detecting release of medicament from the cassette, wherein said release detector transmits release data to the electronic data management system.

Suitably, the device additionally comprises a shake detector for detecting shaking of the medicament container (e.g. prior to actuation of the dispensing mechanism), wherein said shake detector transmits shake data to the electronic data management system.

Suitably, any actuation detector, release detector, or shake detector comprises a sensor for detecting any suitable parameter such as movement. Any suitable sensors are envisaged including the use of optical sensors. The release detector may sense any parameter affected by release of the medicament such as pressure, temperature, sound, moisture, carbon dioxide concentration and oxygen concentration.

Suitably, the medicament dispenser is actuable in response to the inward breath of a patient and includes a breath sensor of any suitable type (e.g. mechanical or electronic) for detecting that inward breath wherein optionally, the sensor communicates with an electronic control system. Thus, for example, in use the patient breathes in through the dispenser (e.g. through the mouthpiece); the breath is detected by the breath sensor; the sensor communicates with the electronic control system to convey an ‘inward breath detected’ signal; and the electronic control system responds by releasing medicament from one or more of the medicament containers for inhalation by the patient.

Suitably, the device additionally comprises a breath trigger for triggering one or all of the component medicament dispensers, said breath trigger being actuable in response to a trigger signal from the electronic data management system. Preferably, the electronic data management system includes a predictive algorithm or look-up table for deriving from the breath data when to transmit the trigger signal. For example, a real-time analysis of the patient breath waveform may be made and the trigger point derived by reference to that analysed waveform.

Suitably, the electronic data management system includes a predictive algorithm or look-up table for calculating the optimum amount of medicament to dispense.

Suitably, the memory on the electronic data management system includes a dose memory for storing dosage data and reference is made to the dose memory in calculating the optimum amount of medicament to dispense.

Suitably, the device additionally comprises a selector for selecting the amount of medicament to dispense from said dispensing mechanism. In one aspect, the selector is manually operable. In another aspect, the selector is operable in response to a signal from the transmitter on the electronic data management system.

Suitably, the device comprises in association with a body or housing thereof, a first transceiver for transmitting and receiving data and in association with the medicament container, a second transceiver for transmitting and receiving data, wherein data is transferable in two-way fashion, from the first transceiver to the second transceiver. The data is preferably in digital form and suitable for transfer by electronic or optical means. A medicament dispenser of this general type is described in pending UK Patent Application No. 0020538.5.

One advantage of embodiments of this type is the ability to store many types of information in different parts of the memory structure of the transceivers. The information is furthermore stored in a form which is readily and accurately transferable. The information could for example, include manufacturing and distribution compliance information written to the memory at various points in the manufacturing or distribution process, thereby providing a detailed and readily accessible product history of the dispenser. Such product history information may, for example, be referred to in the event of a product recall. The compliance information could, for example, include date and time stamps. The information could also include a unique serial number stored in encrypted form or in a password protectable part of the memory which uniquely identifies the product and therefore may assist in the detection and prevention of counterfeiting. The information could also include basic product information such as the nature of the medicament and dosing information, customer information such as the name of the intended customer, and distribution information such as the intended product destination.

On loading or reloading the device with a medicament dispenser or ‘refill’ the second transceiver may, for example, read the unique serial number, batch code and expiry date of the medicament and any other information on the second transceiver. In this way the nature and concentration of the medicament, together with the number of doses used or remaining within the cassette, may be determined. This information can be displayed to the patient on a visual display unit. Other information, such as the number of times the medicament dispenser has been reloaded with a cassette, may also be displayed.

Similarly, should the cassette be removed from the holder before the supply of medicament is exhausted, the same data can be read from the second transceiver and the number of doses remaining or used determined. Other information, such as the date and time of administration of the drug, or environmental exposure data such as the minimum/maximum temperatures or levels of humidity the cassette has been exposed to, may also be read and displayed to the user.

In the event that the supply of medicament within any medicament container becomes exhausted, or that the shelf life of the medicament has expired, or that the first transceiver does not recognise the batch code on the second transceiver, activation of the dispenser may be prevented to safeguard the user. Activation may also be prevented if the medicament has been exposed to extreme environmental conditions for periods outwith the manufacturer's guidelines.

Data may be transferred to and from any transceiver during the period of use of the medicament dispenser by the patient. For example, the medicament dispenser may include an electronic data management system having various sensors associated therewith. Any data collected by the sensors or from any data collection system associated with the electronic data management system including a clock or other date/time recorder is transferable.

Data may be transferred each time the patient uses the device. Or alternatively, data may be stored in a database memory of the electronic data management system and periodically downloaded to any transceiver. In either case, a history of the usage of the device may be built up in the memory of a transceiver.

In one embodiment herein, a history of the usage of the device is transferred to the second transceiver. When the medicament carriers in the cassette are exhausted it is exchanged by the patient for a new refill cassette. At the point of exchange, which will typically occur at the pharmacy, data may be transferred from the exhausted cassette to the refill and vice-versa. Additionally, usage history data may be read from the refill and transferred to a healthcare data management system for example comprising a network computer system under the control of a healthcare data manager.

Methods are envisaged herein whereby the patent is given some sort of reward for returning the refill and making available the data comprised within the second transceiver. Methods are also envisaged herein whereby the healthcare data manager is charged for either receipt of the data from the second transceiver or for its use for commercial purposes. Any rewards or charging may be arranged electronically. The methods may be enabled by distributed or web-based computer network systems in which any collected data is accessible through a hub on the network. The hub may incorporate various security features to ensure patient confidentiality and to allow selective access to information collected dependent upon level of authorisation. The level of user authorisation may be allocated primarily to safeguard patient confidentiality. Beyond this the level of user authorisation may also be allocated on commercial terms with for example broader access to the database being authorised in return for larger commercial payments.

Suitably, the first and second transceiver each comprise an antenna or equivalent for transmitting or receiving data and connecting thereto a memory. The memory will typically comprise an integrated circuit chip. Either transceiver may be configured to have a memory structure which allows for large amounts of information to be stored thereon. The memory structure can be arranged such that parts of the memory are read-only, being programmed during/after manufacture, other parts are read/write and further parts are password protectable. Initial transfer of information (e.g. on manufacture or one dispensing) to or from any transceiver can be arranged to be readily achievable by the use of a reader which is remote from the medicament dispenser, thereby minimising the need for direct product handling. In further aspects, the reader can be arranged to simultaneously read or write to the memory of multiple transceivers on multiple medicament dispensers.

A suitable power source such as a battery, clockwork energy store, solar cell, fuel cell or kinetics-driven cell will be provided as required to any electronic component herein. The power source may be arranged to be rechargeable or reloadable.

Suitably, data is transferable in two-way fashion between the first and second transceiver without the need for direct physical contact therebetween. Preferably, data is transferable wirelessly between the first and second transceiver.

Suitably, the first transceiver is an active transceiver and the second transceiver is a passive transceiver. The term active is used to mean directly-powered and the term passive is used to mean indirectly-powered.

Suitably, the second transceiver comprises a label or tag comprising an antenna for transmitting or receiving energy; and an integrated circuit chip connecting with said antenna, and the first transceiver comprises a reader for said label or tag. In this case the label or tag is a passive transceiver and the reader is an active transceiver.

Preferably, the reader will not need to be in direct contact with the tag or label to enable the tag or label to be read.

The tag may be used in combination and/or integrated with other traditional product labelling methods including visual text, machine-readable text, bar codes and dot codes.

Suitably, the integrated circuit chip has a read only memory area, a write only memory area, a read/write memory area or combinations thereof.

Suitably, the integrated circuit chip has a one-time programmable memory area. More preferably, the one-time programmable memory area contains a unique serial number.

Suitably, the integrated circuit chip has a preset memory area containing a factory preset, non-changeable, unique data item. The preset memory item is most preferably in encrypted form.

Suitably, the integrated circuit chip has plural memory areas thereon. Suitably, any memory area is password protected.

Suitably, any memory area contains data in encrypted form. Electronic methods of checking identity, error detection and data transfer may also be employed.

In one aspect, the integrated circuit has plural memory areas thereon including a read only memory area containing a unique serial number, which may for example be embedded at the time of manufacture; a read/write memory area which can be made read only once information has been written thereto; and a password protected memory area containing data in encrypted form which data may be of anti-counterfeiting utility.

Suitably, the tag is on a carrier and the carrier is mountable on the body or holder of the medicament dispenser or on the cassette.

In one aspect, the carrier is a flexible label. In another aspect, the carrier is a rigid disc. In a further aspect, the carrier is a rectangular block. In a further aspect, the carrier is a collar ring suitable for mounting to the neck of an aerosol container. Other shapes of carrier are also envisaged.

Suitably, the carrier is mouldable or weldable to the cassette or housing. Suitably, the carrier encases the tag. More preferably, the carrier forms a hermetic seal for the tag.

In one aspect, the carrier comprises an insulating material such as a glass material or, a paper material or an organic polymeric material such as polypropylene. Alternatively, the carrier comprises a ferrite material.

The energy may be in any suitable form including ultrasonic, infrared, radiofrequency, magnetic, optical and laser form. Any suitable channels may be used to channel the energy including fibre optic channels.

In one aspect, the second transceiver comprises a radiofrequency identifier comprising an antenna for transmitting or receiving radiofrequency energy; and an integrated circuit chip connecting with said antenna, and the first transceiver comprises a reader for said radiofrequency identifier. In this case the radiofrequency identifier is a passive transceiver and the reader is an active transceiver. An advantage of radiofrequency identifier technology is that the reader need not be in direct contact with the radiofrequency identifier tag or label to be read.

The radiofrequency identifier can be any known radiofrequency identifier. Such identifiers are sometimes known as radiofrequency transponders or radiofrequency identification (RFID) tags or labels. Suitable radiofrequency identifiers include those sold by Phillips Semiconductors of the Netherlands under the trade marks Hitag and Icode, those sold by Amtech Systems Corporation of the United States of America under the trade mark Intellitag, and those sold by Texas Instruments of the United States of America under the trade mark Tagit.

Suitably, the antenna of the RFID tag is capable of transmitting or receiving radiofrequency energy having a frequency of from 100 kHz to 2.5 GHz. Preferred operating frequencies are selected from 125 kHz, 13.56 MHz and 2.4 GHz.

In one aspect, the second transceiver comprises a magnetic label or tag comprising an antenna for transmitting or receiving magnetic field energy; and an integrated circuit chip connecting with said antenna, and the first transceiver comprises a reader for said magnetic label or tag. In this case the magnetic label or tag is a passive transceiver and the reader is an active transceiver.

A suitable magnetic label or tag comprises plural magnetic elements in mutual association whereby the magnetic elements move relative to each other in response to an interrogating magnetic field. A magnetic label or tag of this type is described in U.S. Pat. No. 4,940,966. Another suitable magnetic label or tag comprises a magnetorestrictive element which is readable by application of an interrogating alternating magnetic field in the presence of a magnetic bias field which results in resonance of the magnetorestrictive elements at different predetermined frequencies. A magnetic label of this type is described in PCT Patent Application No. WO92/12402. Another suitable magnetic label or tag comprising plural discrete magnetically active regions in a linear array is described in PCT Patent Application No. WO96/31790. Suitable magnetic labels and tags include those making use of Programmable Magnetic Resonance (PMR) (trade name) technology.

In another aspect, the second transceiver comprises a microelectronic memory chip and the first transceiver comprises a reader for said microelectronic memory chip. The microelectronic memory chip may comprise an Electrically Erasable Programmable Read Only Memory (EEPROM) chip or a SIM card-type memory chip. In this case the microelectronic memory chip is a passive transceiver and the reader is an active transceiver.

Any transceiver herein, particularly a passive transceiver may be mounted on or encased within any suitable inert carrier. The carrier may comprise a flexible sheet which may in embodiments be capable of receiving printed text thereon.

In one aspect, the first transceiver is integral with the body such that a single unit is comprised. The first transceiver may for example be encased within or moulded to the body.

In another aspect, the first transceiver forms part of a base unit which is reversibly associable with the body. The base unit may for example, form a module receivable by the body such as a snap-in module.

Suitably, the device additionally comprises a communicator for wireless communication with a network computer system to enable transfer of data between the network computer system and the electronic data management system.

Dispensers employing such communicators are described in pending PCT Applications No.s PCT/EP00/09291 (PG3786), PCT/EP00/09293 (PG4029) and PCT/EP00/09292 (PG4159). Preferably, the communicator enables two-way transfer of data between the network computer system and the electronic data management system.

Suitably, the data is communicable between the network computer system and the electronic data management system in encrypted form. All suitable methods of encryption or partial encryption are envisaged. Password protection may also be employed. Suitably, the communicator employs radiofrequency or optical signals.

In one aspect, the communicator communicates via a gateway to the network computer system. In another aspect, the communicator includes a network server (e.g. a web server) such that it may directly communicate with the network.

In a further aspect, the communicator communicates with the gateway via a second communications device. Preferably, the second communications device is a telecommunications device, more preferably a cellular phone or pager. Preferably, the communicator communicates with the second communications device using spread spectrum radiofrequency signals. A suitable spread spectrum protocol is the Bluetooth (trade mark) standard which employs rapid (e.g. 1600 times a second) hopping between plural frequencies (e.g. 79 different frequencies). The protocol may further employ multiple sending of data bits (e.g. sending in triplicate) to reduce interference.

In one aspect, the network computer system comprises a public access network computer system. The Internet is one suitable example of a public access network computer system, wherein the point of access thereto can be any suitable entrypoint including an entrypoint managed by an Internet service provider. The public access network computer system may also form part of a telecommunications system, which may itself be either a traditional copper wire system, a cellular system or an optical network.

In another aspect, the network computer system comprises a private access network computer system. The private access network system may for example, comprise an Intranet or Extranet, which may for example, be maintained by a health service provider or medicament manufacturer. The network may for example include password protection; a firewall; and suitable encryption means.

Preferably, the communicator enables communication with a user-specific network address in the network computer system.

The user-specific network address may be selected from the group consisting of a web-site address, an e-mail address and a file transfer protocol address. Preferably, the user-specific network address is accessible to a remote information source such that information from said remote information source can be made available thereto. More preferably, information from the user-specific network address can be made available to the remote information source.

In one aspect, the remote information source is a medicament prescriber, for example a doctors practice. Information transferred from the medicament prescriber may thus, comprise changes to prescription details, automatic prescription updates or training information. Information transferred to the medicament prescriber may comprise compliance information, that is to say information relating to the patient's compliance with a set prescribing programme. Patient performance information relating for example, to patient-collected diagnostic data may also be transferred to the medicament prescriber. Where the dispenser is an inhaler for dispensing medicament for the relief of respiratory disorders examples of such diagnostic data would include breath cycle data or peak flow data.

In another aspect, the remote information source is a pharmacy. Information transferred from the pharmacy may thus, comprise information relating to the medicament product. Information sent to the pharmacy may thus include prescription requests which have been remotely pre-authorised by the medicament prescriber.

In a further aspect, the remote information source is an emergency assistance provider, for example a hospital accident and emergency service or an emergency helpline or switchboard. The information may thus, comprise a distress or emergency assist signal which requests emergency assistance.

In a further aspect, the remote information source is a manufacturer of medicament or medicament delivery systems. Information transferred to the system may thus, comprise product update information. The system may also be configured to feed information back to the manufacturer relating to system performance.

In a further aspect, the remote information source is a research establishment. In a clinical trial situation, information may thus be transferred relating to the trial protocol and information relating to patient compliance fed back to the research establishment.

In a further aspect, the remote information source is an environmental monitoring station. Information relating to weather, pollen counts and pollution levels may thus be made accessible to the system.

Suitably, the device additionally comprises a geographic positioning system such as a global positioning system or a system which relies on the use of multiple communications signals and a triangulation algorithm.

According to another aspect of the present invention there is provided a medicament pack for use with a multi-component combination medicament product, the pack comprising
a first medicament container containing plural co-formulation compatible medicament components; and
at least one further medicament container, each at least one further medicament container containing at least one co-formulation incompatible medicament component; and
wherein the at least one co-formulation incompatible medicament component is kept separate from the plural co-formulation compatible components.

In one aspect, each pack comprises an elongate form medicament carrier having multiple distinct medicament dose portions carried thereby. Suitably, each elongate form medicament carrier is in the form of a strip or tape. The term medicament carrier is used to define any suitable carrier. In a preferred aspect, the carrier has a blister pack form, but it could also, for example, comprise a carrier onto which medicament has been applied by any suitable process including printing, painting and vacuum occlusion.

The elongate form medicament carrier pack is suitable for use with a dispenser device incorporating release means for releasing a distinct medicament dose portion from each of the plural medicament carriers. The release means can have any suitable form. Where the elongate carrier is in the form of a blister strip, the release means may for example, be a means to rupture or otherwise access the blister. In a particular preferred aspect, where the blister strip is peelably accessible, the release means comprises means for peeling apart the blister strip.

### BRIEF DESCRIPTION OF THE DRAWINGS

The invention will now be described with reference to the accompanying drawings in which:

FIG. 1 shows a perspective view of an elongate form medicament carrier suitable for use in accord with an MDPI dispenser of the present invention;

FIG. 2a shows a sectional plan view of a first MDPI dispenser in accord with the invention;

FIG. 2b shows a perspective view of a detail of the MDPI dispenser of FIG. 2a;

FIG. 3 shows a sectional plan view of a second MDPI dispenser in accord with the invention;

FIG. 4a shows a sectional plan view of a third MDPI dispenser in accord with the invention;

FIG. 4b shows a blown apart, perspective view of the MDPI dispenser of FIG. 4a in which two medicament carrier strips associated therewith are shown removed from the dispenser;

FIG. 5a shows a sectional plan view of a fourth MDPI dispenser in accord with the invention;

FIG. 5b shows a blown apart, perspective view of the MDPI dispenser of FIG. 5a in which the strip form, dual series medicament carrier associated therewith is shown removed from the dispenser;

FIGS. 6a and 6b respectively show schematic top and bottom views of a reservoir DPI dispenser in accord with the present invention, wherein the mouthpiece is in the storage position;

FIGS. 6c and 6d respectively show schematic top and bottom views of the reservoir DPI dispenser of FIGS. 6a and 6b, wherein the mouthpiece is in the in-use position;

FIGS. 7a and 7b respectively show upper and lower sectional side views of a second reservoir DPI dispenser in accord with the present invention, wherein the mouthpiece is in the storage position;

FIGS. 7c and 7d respectively show upper and lower sectional side views of the reservoir DPI dispenser of FIGS. 7a and 7b, wherein the mouthpiece is in the in-use position;

FIGS. 8a to 8c show a third reservoir DPI dispenser herein respectively in perspective, exploded (part cut-away) and sectional side views;

FIGS. 9a to 9c show a first UDPI capsule dispenser herein respectively in perspective, exploded and sectional side views;

FIGS. 10a to 10c show a second UDPI capsule dispenser herein respectively in perspective, exploded and sectional side views;

FIGS. 11a and 11b show a fourth MDPI dispenser herein respectively in exploded perspective and side views; and

FIG. 12a shows a perspective view of a dual MDI dispenser herein and FIG. 12b shows the dispenser of FIG. 12a in part cut-away view.

### DETAILED DESCRIPTION OF THE DRAWINGS

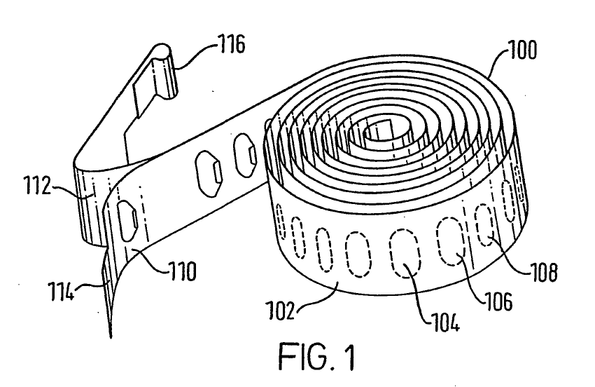

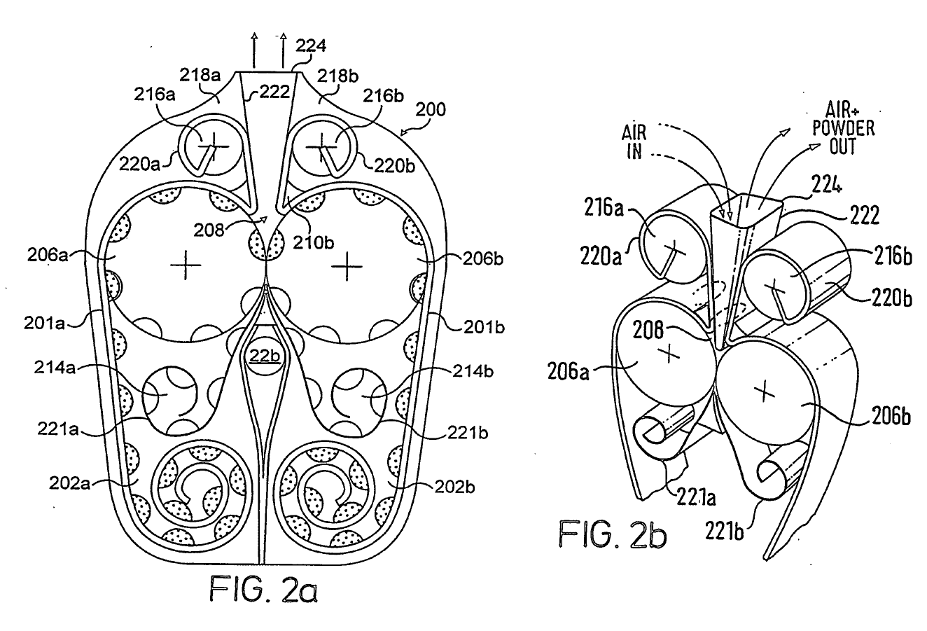

All of the Figures herein show details of suitable medicament dispensers (with the exception of FIG. 1 which shows a suitable medicament carrier) in accord with the invention. In use, the first medicament container of each dispenser of FIGS. 2a to 12b contains a medicament formulation comprising containing plural co-formulation compatible medicament components; and the second medicament container of each dispenser comprises a second medicament formulation comprising at least one co-formulation incompatible medicament component.

FIG. 1 shows a medicament carrier 100 suitable for use in an MDPI type dispenser in accord with the present invention. The medicament carrier comprises a flexible strip 102 defining a plurality of pockets 104, 106, 108 each of which contains a portion of a dose of active medicament of a form suitable for inhalation and in the form of powder. In accord with the present invention, two such strips 102, one containing the first active medicament and the other containing the second active medicament are employed in a single medicament dispenser, wherein each strip provides the component active medicament dose portions of the combination medicament product. Each strip may be of the same size and/or contain the same dose amount (e.g. volume or mass) or in alternative embodiments, strips of different sizes and/or containing different dose amounts may be employed in combination.

The strip comprises a base sheet 110 in which blisters are formed to define the pockets 104, 106, 108 and a lid sheet 112 which is hermetically sealed to the base sheet except in the region of the blisters in such a manner that the lid sheet 112 and the base sheet 110 can be peeled apart. The sheets 110, 112 are sealed to one another over their whole width except for the leading end portions 114, 116 where they are preferably not sealed to one another at all.

The lid 112 and base 110 sheets are each formed of a plastics/aluminium laminate and are suitably adhered to one another by heat sealing. The lid sheet 112 comprises at least the following successive layers: (a) paper; adhesively bonded to (b) polyester; adhesively bonded to (c) aluminium foil; that is coated with a heat seal lacquer for bonding to the base sheet. The base sheet 110 comprises at least the following successive layers: (a) oriented polyamide (OPA); adhesively bonded to (b) aluminium foil; adhesively bonded to (c) a third layer comprising a polymeric material (e.g. polyvinyl chloride).

The strip 102 is shown as having elongate pockets 104, 106, 108 which run transversely with respect to the length of the strip 102. This is convenient in that it enables a large number of pockets 104, 106, 108 to be provided in series arrangement along a given strip 102 length. The strip 102 may, for example, be provided with sixty or one hundred pockets but it will be understood that the strip 102 may have any suitable number of pockets.

FIG. 2a illustrates the base unit 200 of a medicament dispenser according to the invention. In use, a cover (not shown) would be provided to the base unit 200. First and second medicament-containing blister strips 201a, 201b are positioned within respective left and right chambers 202a, 202b of the base unit 200. The first blister strip 201a contains multiple dose portions of a first active medicament component. The second blister strip 201b contains multiple dose portions of a second active medicament component. Each blister strip 201a, 201b engages a respective multi-pocket index wheel 206a, 206b, and successive pockets are thereby guided towards a commonly located opening station 208. The rotation of the index wheels 206a, 206b is coupled. At the opening station 208, the lid foil 220a, 220b and base foil 221a, 221b parts of each strip 201a, 201b are peelably separable about a beak 210a, 210b. The resulting empty base foil 221a, 221b coils up in respective base take-up chambers 214a, 214b. The used lid foil 220a, 220b is fed over its respective beak 210a, 210b and coiled about a lid take-up spindle 216a, 216b in the lid take-up chamber 218a, 218b.

Released powder form medicament from both the first 201a and second 201b strips is channelled via common manifold 222 to a single outlet 224 for inhalation by the patient. Importantly, the dispenser thereby enables different medicament types to be stored separately in each of the strips 201a, 201b but the release and delivery thereof to the patient as a combined inhaled product.

FIG. 2b shows the release of medicament in more detail. The patient breathes in through the outlet 224 resulting in negative pressure being transmitted through manifold 222 to the opened pockets of the strips 201a, 201b at the opening station 208. This results in the creation of a venturi effect which results in the powder contained within each of the opened pockets 201a, 201b being drawn out through the common manifold 222 to the outlet 224 and hence to the patient. Mixing of each separately delivered component of the combined medicament product will thus happens during the delivery process, particularly as a result of the so created venturi effect.

The dispenser is actuated by pressing a button on the side of the dispenser (not shown) which actuates a DC motor 226 to index the internal mechanism by one pocket of medicament for each blister strip 201a, 201b. The DC motor 226, thus results in indexing of each strip 201a, 201b and coiling up of the waste foils.

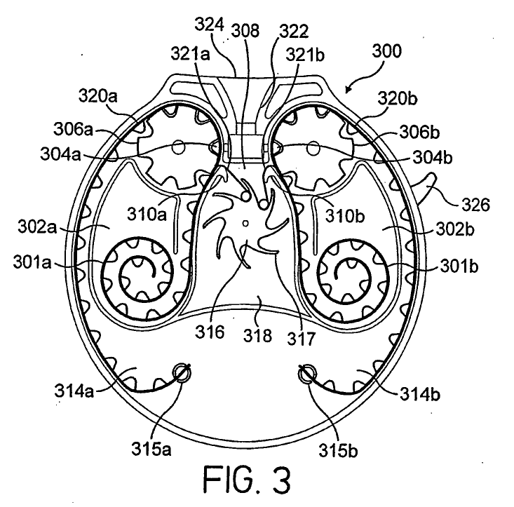

FIG. 3 illustrates a sectional view of base unit 300 of a medicament dispenser according to the invention. In use, a protective cover (not shown) would be provided to the base unit 300. First and second medicament-containing blister strips 301a, 301b are positioned within respective left and right chambers 302a, 302b of the base unit 300. The first blister strip 301a contains multiple dose portions of a first active medicament component. The second blister strip 301b contains multiple dose portions of a second active medicament component. Each blister strip 301a, 301b engages in respective multi-pocket index wheel 306a, 306b, and successive pockets are thereby guided towards a central opening station 308. The rotation of the index wheels 306a, 306b is optionally coupled together. At the opening station 308, the lid foil 320a, 320b and base foil 321a, 321b parts of each strip 301a, 301b are peelably separable about beak 310a, 310b. The resulting empty base foil 321a, 321b coils up in respective base take-up chambers 314a, 314b. A base foil anchor 315a, 315b anchors the end of each respective base foil 321a, 321b in its chamber 314a, 314b. The used lid foil 320a, 320b feeds over its respective beak 310a, 310b and coils about common lid take-up spindle 316 in the common lid take-up chamber 318.

It will be noted that common lid take-up spindle 316 comprises plural arms 317 that splay out radially from the centre to give it an overall ‘collapsible wheel’ form. In use, as lid-foil 320a, 320b wraps around the spindle 316, the arms 317 collapse inwardly hereby reducing the diameter of the spindle 316 itself but acting to maintain a roughly constant effective winding diameter as defined by the diameter of the spindle 316 in combination with the used lid foil 320a, 320b wrapped there around. The maintenance of this constant effective winding diameter ensures uniform indexing of each strip 301a, 301b over the entire strip length.

In use, the dispenser is primed by actuating lever 326 located on the side of the dispenser to drivably actuate the lid-take up spindle 316 to advance each blister strip 301a, 301b, thereby causing the leading pocket 304a, 304b thereof to be peeled open. To access the contents of the opened pockets 304a, 304b, the patient then breathes in through the outlet 324. This results in negative pressure being transmitted through manifold 322 to the opened leading pocket 304a, 304b of each strip 301a, 301b at the opening station 308. This in turn, results in the medicament powder contained within each of the opened pockets 304a, 304b being drawn out through the common manifold 322 to the outlet 324 and hence to the patient as an inhaled combination medicament dose. It be appreciated that, mixing of each separately delivered component of the combined medicament product happens as the powder is transported from each opened pocket 304a, 304b to the outlet 324.

Importantly, the dispenser of FIG. 3 enables different medicament formulation types to be stored separately in each of the strips 301a, 301b but allows for the release and delivery thereof to the patient via the single outlet 324 as a combined inhaled product.

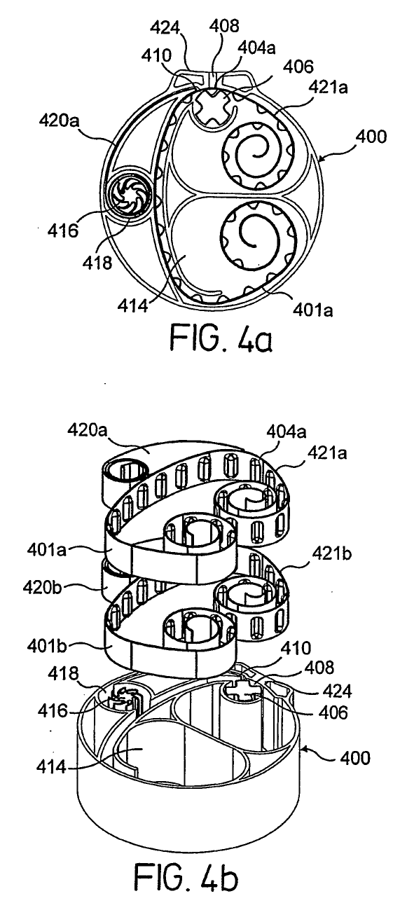

FIGS. 4a and 4b respectively illustrate sectional and perspective views of base unit 400 of a medicament dispenser according to the invention. In use, a protective cover (not shown) would be provided to the base unit 400. First and second medicament-containing blister strips 401a, 401b are positioned one on top of the other (in ‘double-decker’ configuration) in the base unit 400. The first blister strip 401a contains multiple dose portions of a medicament formulation comprising containing plural co-formulation compatible medicament components. The second blister strip 401b contains multiple dose portions of a second medicament formulation comprising at least one co-formulation incompatible medicament component. In this configuration, each blister strip 401a, 401b shares the same internal mechanism elements (e.g. drive, index, opening) of the base unit 400. Thus, each strip 401a, 401b engages shared multi-pocket index wheel 406 and successive pockets are thereby guided towards a central opening station 408. At the opening station 408, the lid foil 420a, 420b and base foil 421a, 421b parts of each strip 401a, 401b are peelably separable about beak 410. The resulting empty base foil 421a, 421b coils up in base take-up chamber 414. The used lid foil 420a, 420b feeds over beak 410 and coils about common ‘collapsible wheel’ form lid take-up spindle 416 in the common lid take-up chamber 418.

In use, the dispenser is primed by drivably actuating the lid-take up spindle 416 to advance each blister strip 401a, 401b, thereby causing the leading pocket 404a (leading pocket not visible on second strip) thereof to be peeled open. To access the contents of the opened pocket 404a the patient then breathes in through the outlet 424. This results in negative pressure being transmitted to the opened leading pockets 404a at the opening station 408. This in turn, results in the medicament powder contained within the opened pocket 404a of each strip 401a, 401b being drawn out to the outlet 424 and hence to the patient as an inhaled combination medicament dose.

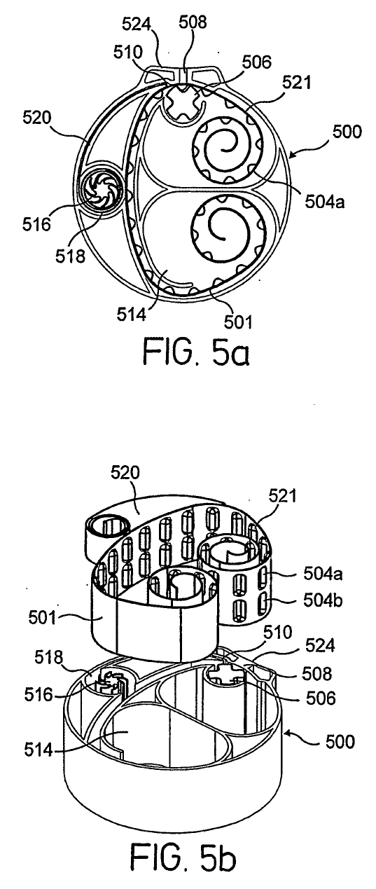

FIGS. 5a and 5b respectively illustrate sectional and perspective views of base unit 500 of a medicament dispenser that may be appreciated to be a variation of the dispenser of FIG. 4. In the dispenser of FIG. 4, the ‘double decker’ configuration of separate strips 501a, 501b of FIG. 4 is replaced by a single strip 401 comprising dual series of pockets 404a, 404b arranged in parallel fashion thereon for receipt by the base unit 400. The first series of pockets 404a contains multiple dose portions of a medicament formulation comprising containing plural co-formulation compatible medicament components. The second series of pockets 404b contains multiple dose portions of a second medicament formulation comprising at least one co-formulation incompatible medicament component.

As with the dispenser of FIGS. 4a and 4b, each series of blister pockets 404a, 404b shares the same internal mechanism elements (e.g. drive, index, opening) of the base unit 400. Thus, the dual series strip 401 engages multi-pocket index wheel 406 and successive pockets of both series are thereby guided towards a central opening station 408. At the opening station 408, the lid foil 420 and base foil 421 parts of the dual series strip 401 are peelably separable about beak 410. The resulting empty base foil 421 coils up in base foil take-up chamber 414. The used lid 420 feeds over beak 410 and coils about common ‘collapsible wheel’ form lid take-up spindle 416 in the common lid take-up chamber 418.

In use, the dispenser is primed by drivably actuating the lid-take up spindle 416 to advance the dual series blister strip 401 thereby causing the leading pockets 404a, 404b of each series thereof to be peeled open. To access the contents of the opened pockets 404a, 404b the patient then breathes in through the outlet 424. This results in negative pressure being transmitted to the opened leading pockets 404a, 404b at the opening station 408. This in turn, results in the medicament powder contained within each of the opened pockets 404a, 404b being drawn out to the outlet 424 and hence to the patient as an inhaled combination medicament dose.

As shown in FIG. 5, the pockets of each series are of equivalent size and shape. It will be appreciated, that in variations, the pockets of one series may be shaped and/or sized differently from that of another series.

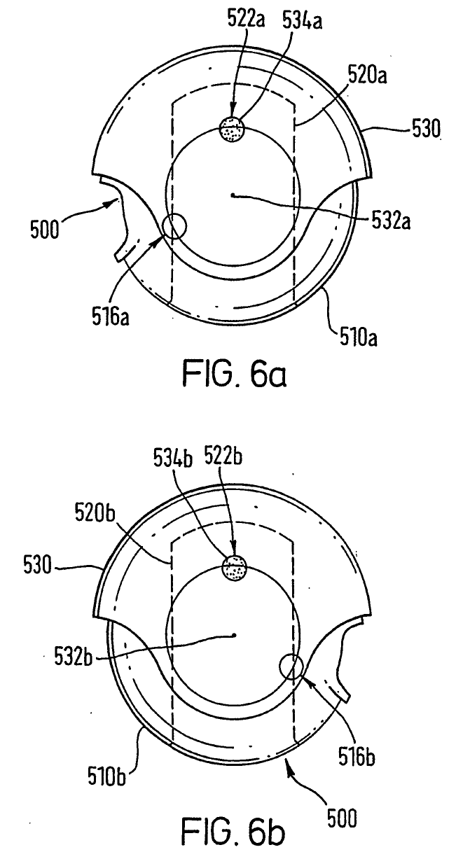

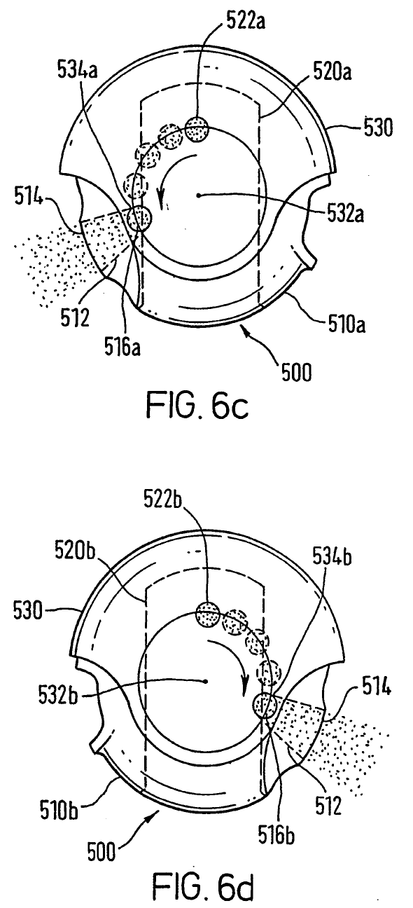

FIGS. 6a and 6b respectively show top and bottom views of a reservoir DPI dispenser herein in a non-dispensing position. FIGS. 6c and 6d respectively show top and bottom views of the dispenser in a dispensing position. The dispenser comprises a generally circular body 500 comprised of two clamshell halves 510a, 510b mating together to define a common outlet 512 defining a mouthpiece 514 (only visible in FIGS. 6c and 6d). Each clamshell half 510a, 510b is also provided with a dispensing orifice 516a, 516b which in the open position communicates with the common outlet 512 and mouthpiece 514 for dispensing of medicament therethrough. Defined by each clamshell half 510a, 510b there is a respective medicament container 520a, 520b for containment of dry powder medicament. The first medicament container 520a contains a medicament formulation comprising containing plural co-formulation compatible medicament components. The second medicament container 520b contains a second medicament formulation comprising at least one co-formulation incompatible medicament component. Each container 520a, 520b is provided with a delivery orifice 522a, 522b for delivery of its dry powder medicament contents. A unitary cover 530 is also provided to the body 500 wherein the cover is pivotally mounted at pivot points 532a, 532b to the clamshell halves 510a, 510b of body 500 such that it is rotatable by 180° around the body 500. The inner part of the cover 530 is provided with both top and bottom metering recesses 534a, 534b. In the cover closed position, each metering recess 534a, 534b locates adjacent the delivery orifice 522a, 522b of its respective medicament container 520a, 520b such that powder may enter each metering recess 534a, 534b therefrom. In the open position, each metering recess 534a, 534b locates adjacent its respective dispensing orifice 516a, 516b such that powder may pass therethrough to the common outlet 512 and mouthpiece 514.

It may be appreciated that the device is operable by a single-handed operation in which the cover 530 is held in the cupped fingers (not shown) of a user and the body 500 rotated through 180° by a thumb action of the user to bring the device from the open to closed position and vice versa. It may also be appreciated that the 180° rotation of the cover 530 acts such as to either expose or cover the mouthpiece 514 and to move each metering recess 534a, 534b from a loading position to a dispensing position.

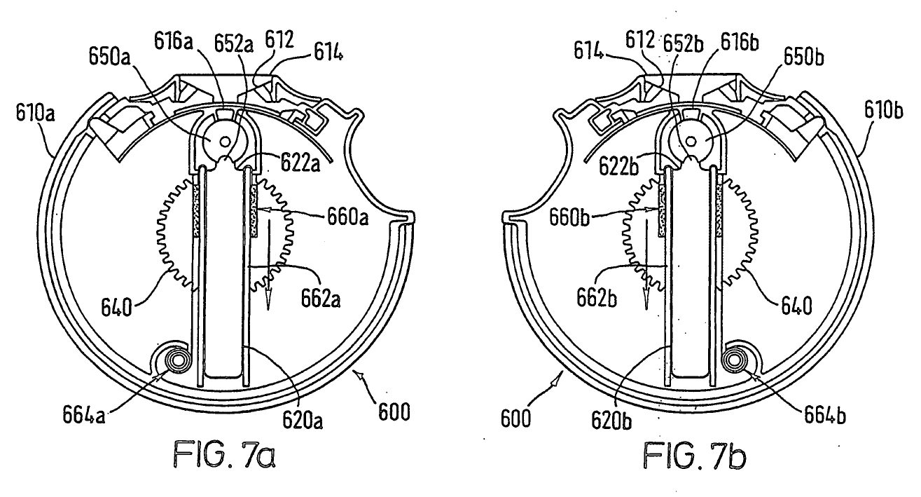

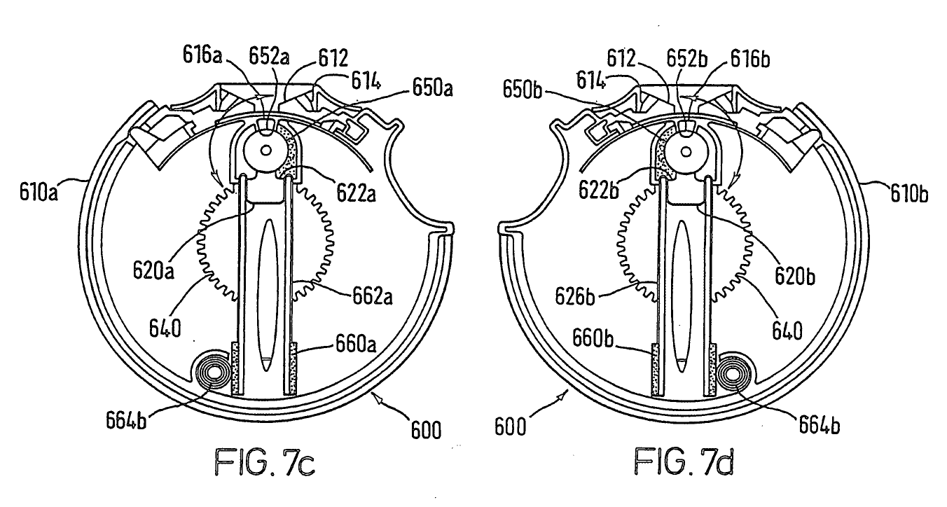

FIGS. 7a and 7b respectively show top and bottom views of another reservoir DPI dispenser herein in a non-dispensing position. FIGS. 7c and 7d respectively show top and bottom views of the dispenser in a dispensing position. The dispenser comprises a generally circular body 600 comprised of two clamshell halves 610a, 610b mating together to define a common outlet 612 defining a mouthpiece 614. Each clamshell half 610a, 610b is also provided with a dispensing orifice 616a, 616b that in the open position communicates with the common outlet 612 and mouthpiece 614 for dispensing of medicament therethrough. Provided within each clamshell half 610a, 610b there is a respective collapsible tube container 620a, 620b for containment of dry powder medicament. The first medicament container 620a contains a medicament formulation comprising containing plural co-formulation compatible medicament components. The second medicament container 620b contains a second medicament formulation comprising at least one co-formulation incompatible medicament component. Each container 620a, 620b is provided with a delivery orifice 622a, 622b for delivery of powder. A cover (not visible) is provided to the body 600 wherein the cover is pivotally mounted such that it is rotatable by 180° around the body 600. The cover is co-axially mounted and rotationally coupled to common drive wheel 140 such that rotation of the cover results in rotation of the drive wheel 640. The common drive wheel 640 engages first and second metering wheels 650a, 650b each of which is provided with a metering recess 652a, 652b. In the cover-closed position, each metering recess 652a, 652b locates adjacent the delivery orifice 622a, 622b of its respective medicament cartridge 620a, 620b such that powder may enter each metering recess 652a, 652b therefrom. In the open position, each metering recess 652a, 652b locates adjacent its respective dispensing orifice 616a, 616b such that powder may pass therethrough to the common outlet 612 and mouthpiece 614.

It may be seen that each powder container 620a, 620b is also provided with a system for ensuring constant delivery of powder to its delivery orifice 622a, 622b. The system comprises a collar 660a, 660b movable along a track 662a, 662b located on either side of the tubular container 620a, 620b. Pulling force is applied to the collar 660a, 660b by constant force spring 664a, 664b. As each collar 660a, 660b is pulled along its track 662a, 662b by the action of its spring 664a, 664b the tube 620a, 620b is squeezed and powder is urged towards its delivery orifice 622a, 622b.

It may be appreciated that the dispenser of FIGS. 7a to 7d is operable by a single-handed operation in which the cover (not shown) is held in the cupped fingers (not shown) of a user and the body 600 rotated through 180° by a thumb action of the user to bring the device from the open to closed position and vice versa. It may also be appreciated that the 180° rotation of the cover acts such as to both expose or cover the mouthpiece 614 and move each metering recess 652a, 652b from a loading position to a dispensing position.

In a variation of the dispenser of FIGS. 7a to 7d, the common drive wheel 640 is replaced by first and second independent drive wheels, each of which drives respective first and second metering wheels 650a, 650b. In this variation, the movement of each independent drive wheel is independently and releasably couplable to the movement of the cover. In one mode of use, only one independent drive wheel is coupled to the cover such that cover movement results only in movement of that drive wheel and its corresponding metering wheel 650a such that powder is metered from only one medicament container 620a. In another mode of use, both independent drive wheels are coupled to the cover such that cover movement results in movement both drive wheels and corresponding metering wheels 650a, 650b such that powder is metered from both medicament containers 620a, 620b. In this variation, the dispenser may thus be arranged to deliver only one medicament powder or two medicament powders as a combination product.

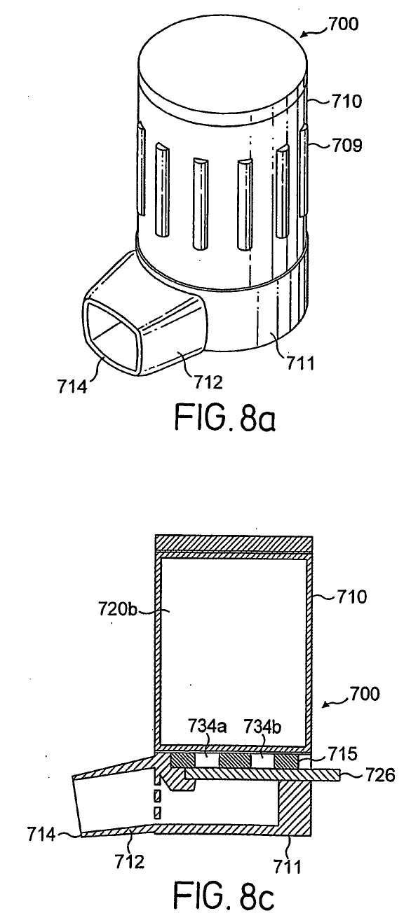

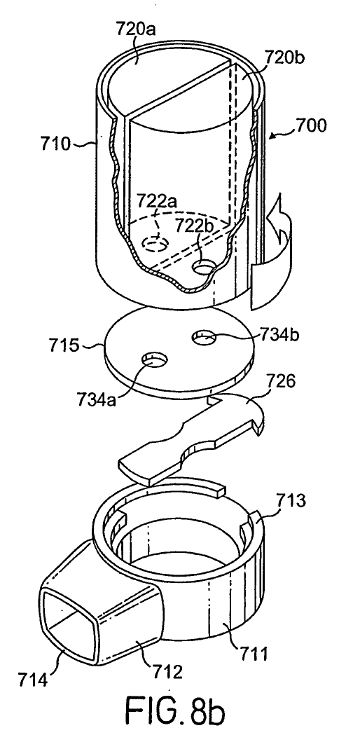

FIGS. 8a to 8c illustrate a third reservoir dispenser herein, as shown respectively in perspective, exploded and sectional side views. The dispenser comprises a generally L-shaped body 700 comprised of upper column-shaped housing 710 rotationally mounted to base 711. The base 711 is shaped to define a common outlet 712 in the form of a mouthpiece 714. The column-shaped housing 710 has grips 709 for ease of patient grip, and is provided with two medicament containers 720a, 720b (both visible in FIG. 8b only) of semi-circular cross-section, each for containment of dry powder medicament. The first medicament container 720a contains a medicament formulation comprising containing plural co-formulation compatible medicament components. The second medicament container 720b contains a second medicament formulation comprising at least one co-formulation incompatible medicament component. Each container 720a, 720b is itself provided with circular delivery orifice 722a, 722b for delivery of its dry powder medicament contents. Locating within the upper rim 713 of the base 711 and fixedly mounted with respect thereto, there is provided circular plate 715. The plate has two circular metering orifices 734a, 734b, each sized and shaped to register with the circular delivery orifices 722a, 722b of the respective containers 720a, 720b, in a metering position. Dispensing lever 726 locates beneath the plate 715 and is mounted for rotation with respect to the base 711. The Lever is rotationally movable from a non-dispensing position in which it acts to close off communication between the metering orifices 734a, 734b of the plate 715 to a dispensing position in which the metering orifices 734a, 734b communicate with the common outlet 712 and mouthpiece 714 of the base 711 for dispensing of medicament therethrough.

Usage of the dispenser of FIGS. 8a to 8c involves two distinct actions, namely metering and dispensing. In the metering action, the column 710 is rotated with respect to the base 711 until the circular delivery orifices 722a, 722b of the respective containers 720a, 720b are brought into registration with the circular metering orifices 734a, 734b of the plate 715. A metered quantity of the medicament powder contents of each container 720a, 720b is thereby delivered under gravity to each metering orifice 734a, 734b. The column 711 is then rotated in a reverse sense to bring the respective orifices 722a, 722b and 734a, 734b out of registration with each other but leaving a metered quantity of medicament powder in each metering orifice 734a, 734b. It will be appreciated that in the metering stage, the lever 726 is in the non-dispensing (i.e. closed off) position with respect to the plate 715.

In the dispensing action, the lever 726 is now rotated from the non-dispensing position in which it acts to close off communication between the metering orifices 734a, 734b of the plate 715 to the dispensing position in which the volume of medicament powder contained within each metering orifice 734a, 734b is released to the base for dispensing to an inhaling patient through the common outlet 712 and mouthpiece 714.

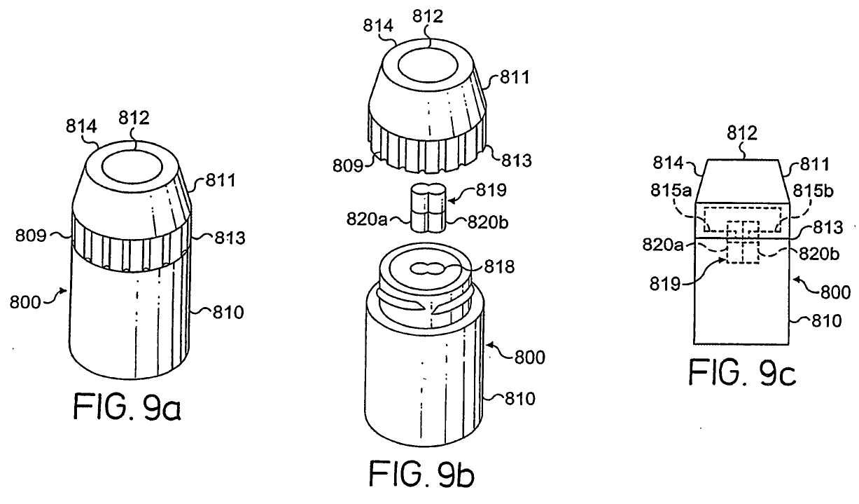

FIGS. 9a to 9c illustrate a first DPI capsule dispenser herein, as shown respectively in perspective, exploded and sectional side views. The dispenser comprises a generally cylindrical body 800 comprised of column housing 810 rotationally mounted to dispensing head 811. The head 811 is shaped to define a common outlet 812 in the form of a mouthpiece 814 and has grips 809 for ease of patient grip thereof. The housing 810 is provided with dual-lobed cavity 818 shaped for receipt of dual-lobed capsule body 819, which defines two separate medicament containers 820a, 820b, each for containment of dry powder medicament. In essence, the dual-lobed capsule 819 acts as a simple type of ‘refill cassette’ comprising dual medicament containers 820a, 820b. The first medicament container 820a contains a unit dose portion of a medicament formulation comprising containing plural co-formulation compatible medicament components. The second medicament container 820b contains a unit dose portion of a second medicament formulation comprising at least one co-formulation incompatible medicament component. Located at the mating rim 813 of the head 811 and fixedly mounted with respect thereto, there are provided two jutting inner edge features 815a, 815b at 180° rotational spacing relative to each other. The head 811 is rotatable relative to the housing from a first position in which the jutting inner edges 815a, 815b thereof are distant from the dual-lobed capsule 819 to a second position in which the edges 815a, 815b destructively interact with the capsule to sever each respective container part 820a, 820b thereof. Once the capsule is severed, the medicament powder held within each container 820a, 820b is made available for inhalation by a patient through the common outlet 812 and mouthpiece 814.

Usage of the dispenser of FIGS. 9a to 9c involves accessing of the capsules and then dispensing the contents thereof to a patient. In the capsule accessing action, the housing 810 is rotated with respect to the head 811 until the jutting edges 815a, 815b sever the dual-lobed capsule 819 to enable access to contents of the medicament containers 820a, 820b. The patent then inhales through the mouthpiece 814 to aerosolise the powder contained in each container 820a, 820b of the capsule 819 for delivery as a combination product through the common outlet 812 and mouthpiece 814.

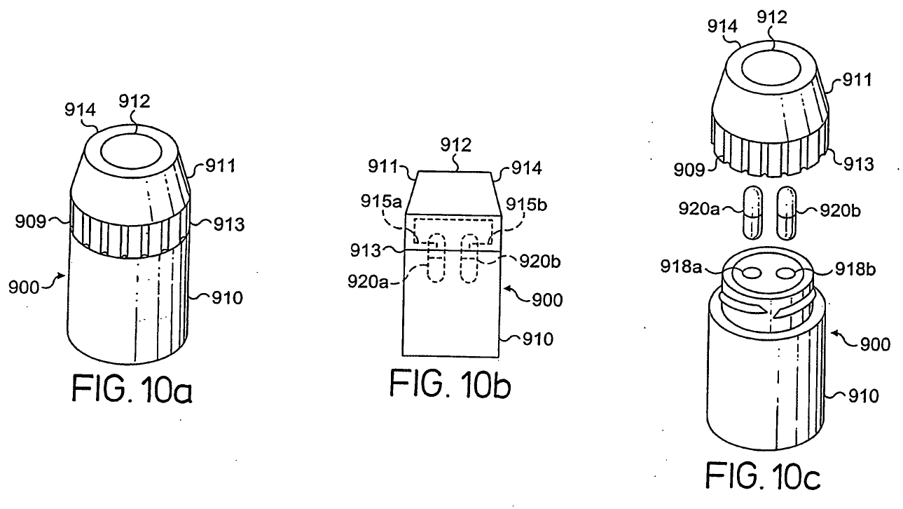

FIGS. 10a to 10c illustrate a second DPI capsule dispenser herein, as shown respectively in perspective, exploded and sectional side views. It will be appreciated that this dispenser is a dual capsule variation of the first DPI dispenser shown in FIGS. 9a to 9c. The dispenser comprises a generally cylindrical body 900 comprised of column housing 910 rotationally mounted to dispensing head 911. The head 911 is shaped to define a common outlet 912 in the form of a mouthpiece 914 and has grips 909 for ease of patient grip thereof. The housing 910 is provided with dual cavities 918a, 918b, each shaped for receipt of a medicament container 920a, 920b in the form of a capsule for containing dry powder medicament. The first medicament container 920a contains a unit dose portion of a medicament formulation comprising containing plural co-formulation compatible medicament components. The second medicament container 920b contains a unit dose portion of a second medicament formulation comprising at least one co-formulation incompatible medicament component. Located at the mating rim 913 of the head 911 and fixedly mounted with respect thereto, there are provided two jutting inner edge features 915a, 915b at 180° rotational spacing relative to each other. The head 911 is rotatable relative to the housing from a first position in which the jutting inner edges 915a, 915b thereof are distant from respective capsules 920a, 920b to a second position in which each edge 915a, 915b destructively interacts with a capsule 920a, 920b to sever it open Once each capsule 920a, 920b is severed, the medicament powder held within it is made available for inhalation by a patient through the common outlet 912 and mouthpiece 914.

Usage of the dispenser of FIGS. 10a to 10c involves accessing of the capsules and then dispensing the contents thereof to a patent. In the capsule accessing action, the housing 910 is rotated with respect to the head 911 until the jutting edges 915a, 915b sever the capsule 920a, 920b to enable access to the dry powder medicament contents thereof. The patient then inhales through the mouthpiece 914 to aerosolise the powder contained in each capsule 920a, 920b for delivery as a combination product through the common outlet 912 and mouthpiece 914.

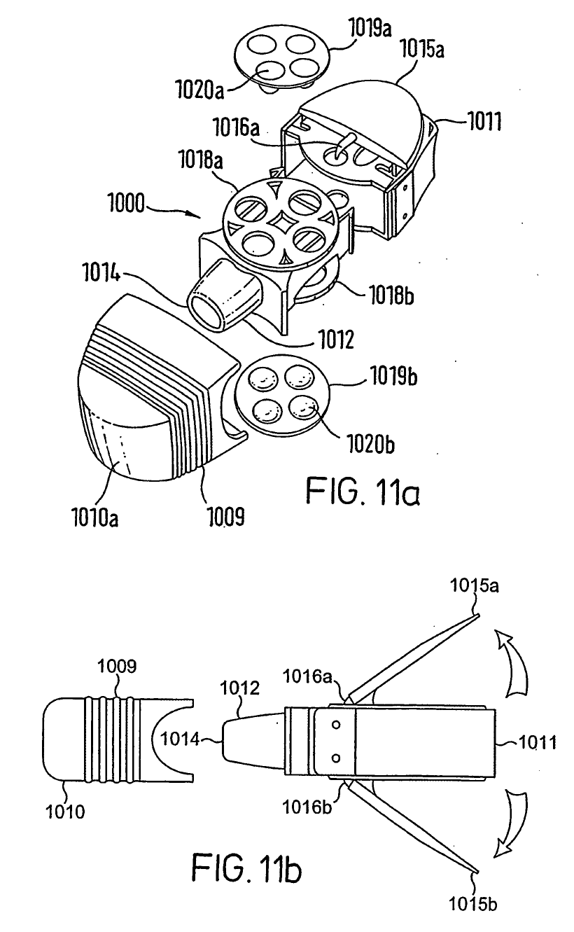

FIGS. 11a and 11b show a fourth MDPI dispenser herein respectively in exploded perspective and side views. The dispenser comprises a central body 1000 comprising upper and lower, rotationally mounted circular carriages 1018a, 1018b, each shaped for receipt of a circular medicament carrier disk 1019a, 1019b. Each disk 1019a, 1019b has provided thereto four, evenly spaced blisters 1020a, 1020b for containing medicament powder. The first blister pack 1019a contains multiple dose portions of a medicament formulation comprising containing plural co-formulation compatible medicament components. The second blister pack 1019b contains multiple dose portions of a second medicament formulation comprising at least one co-formulation incompatible medicament component. Variations involving, for example, six and eight blisters disks 1019a, 1019b are also envisaged. The body 1000 is also provided with a common outlet 1012 defining a mouthpiece 1014.

The body 1000 of the dispenser is housed within an elongate housing comprised of two mating halves 1010, 1011. One half 1010 acts as a cover for the mouthpiece 1014 and is provided with finger grips 1009 for ease of its removal by a patient. The second half 1011 is provided with upper and lower, hingedly mounted wings 1015a, 1015b. The tip of each wing 1015a, 1015b is provided with a piercing element 1016a, 1016b for enabling piercable access to a blister 1020a, 1020b of an associated disk 1019a, 1019b, thereby enabling release of dry powder medicament therefrom. Once so released, the dry powder is made available for inhalation by a patient via the common outlet 1012 and mouthpiece 1014.

In use, each carriage 1018a, 1018b is first rotated to bring an unopened blister 1020a, 1020b of each disk 1019a, 1019b to a position where it may be piercably accessed. The upper and lower wings 1015a, 1015b are then squeezed towards each other to bring the piercing element 1016a, 1016b of each into piercing contact with the blister 1020a, 1020b thereby piercing it open. The patient then inhales through the mouthpiece 1014 to aerosolise the dry powder medicament contents of each opened blister 1020a, 1020b and draw such contents via common outlet 1012 and mouthpiece 1014 for inhalation as a combination product.

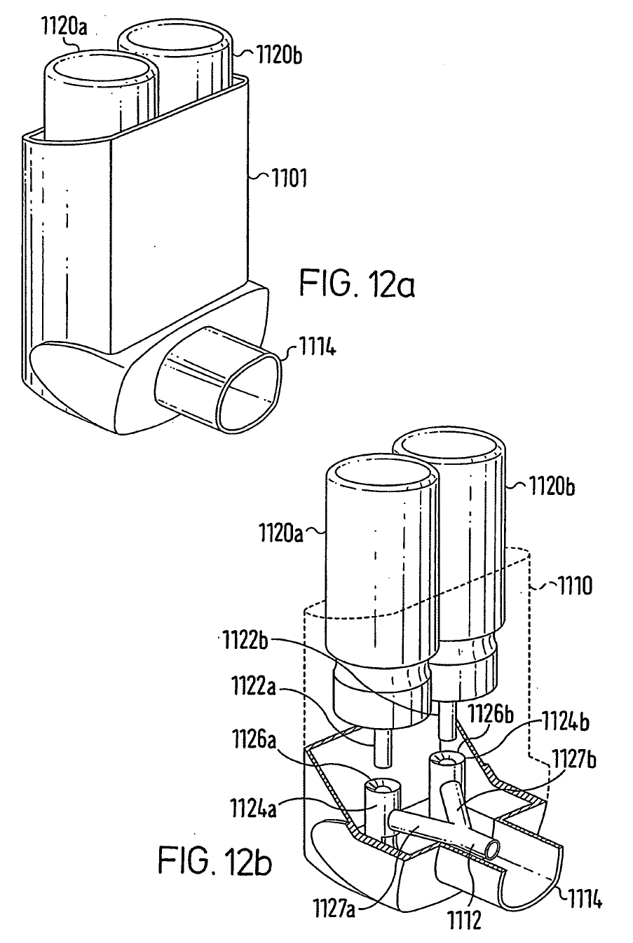

FIG. 12a shows a perspective view of a dual MDI dispenser herein and FIG. 12b shows the dispenser of FIG. 12a in part cut-away view. The dual MDI dispenser comprises a generally L-shaped tubular housing 1101 shaped for receipt of two aerosol containers 1120a, 1120b. The first aerosol container 1120a contains a medicament formulation comprising containing plural co-formulation compatible medicament components in aerosol formulation form. The second aerosol container 1120b contains a second medicament formulation comprising at least one co-formulation incompatible medicament component in aerosol formulation form. The housing is open at one end (which will hereinafter be considered to be the top of the device for convenience of description) and is closed at the other. A common mouthpiece 1114 leads laterally from the closed end of the housing 1101. In variations, the mouthpiece 1114 may if desired, be designed as a nozzle for insertion into the patient's nostril.

Each aerosol container 1120a, 1120b has an outlet valve stem 1122a, 1122b at one end. Each valve 1122a, 1122b can be depressed to release a metered dose from its respective aerosol container 1120a, 1120b. Each aerosol container 1120a, 1120b locates in the housing 1101 such that one end protrudes from its open top. Valve support 1124a, 1124b are provided at the lower end of the housing 1101 and are provided with respective passages 1126a, 1126b in which the valve stem 1122a, 1122b of each respective aerosol container 1120a, 1120b can be located and supported. A second passage 1127a, 1127b leads from each support 1124a, 1124b and is directed towards common outlet passage 1112.

In use, the protruding portion of each aerosol container 1120a, 1120b is depressed to move that container 1120a, 1120b relative to its valve stem 1122a, 1122a to open the valve and discharge an aerosol form dose of medicament through passages 1127a, 1127b to common outlet passage 1112 and thence to the mouthpiece 1114 from which it can be inhaled by a patient. A measurement amount (e.g. a part-combination dose) will be released from each aerosol container 1120a, 1120b each time it is fully depressed.

It will be appreciated that the required depression of each aerosol container 1120a, 1120b is achievable by a dual fingered patient action whilst the base of the housing 1101 is held in a patient's cupped hand. In variations, the movement of the containers 1120a, 1120b may be coupled (e.g. through use of a coupling element) thereby ensuring that both containers 1120a, 1120b are fired in tandem.

In one example, the co-formulation compatible components comprise fluticasone propionate and salmeterol, or a salt thereof (particularly the xinafoate salt) and the co-formulation incompatible component comprises a PDE-4 inhibitor, an anti-cholinergic or a mixture thereof.

In a first particular set of examples, the first medicament formulation of each dispenser shown in FIGS. 2a to 12b comprises fluticasone propionate and salmeterol xinafoate salt and the second medicament formulation comprises a PDE-4 inhibitor, an anti-cholinergic or a mixture thereof.

In a second particular set of examples, the first medicament formulation of each dispenser shown in FIGS. 2a to 12b comprises budesonide and formoterol fumarate and the second medicament formulation comprises a PDE-4 inhibitor, an anti-cholinergic or a mixture thereof.

It may be appreciated that any of the parts of the device or any medicament thereof, which contacts medicament may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

The medicament dispenser device of the invention is suitable for dispensing ‘multi-active’ medicament combinations, particularly for the treatment of respiratory disorders such as asthma and chronic obstructive pulmonary disease (COPD), bronchitis and chest infections.

The overall ‘multi-active’ combination product comprises in combination, a first component comprising plural co-formulation compatible medicament components; and at least one further component comprising at least one co-formulation incompatible medicament component. The dispenser device herein enables the delivery of these components together in combination, even though on containment within the device they are kept separate.

Appropriate medicaments may thus be selected from, for example, analgesics, e.g., codeine, dihydmmorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiInfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2 (3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt), diuretics, e.g., amiloride; anticholinergics, e.g., ipratroplum (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

The medicament dispenser herein is suitable for delivering a combination medicament product. In accord with the present invention, the first medicament container contains two or more co-formulation compatible active medicament components and the at least one further medicament container contains at least one co-formulation incompatible active medicament component. The delivered combination product therefore comprises at least three active medicament components

The at least three active medicament components are suitably selected from the group consisting of anti-inflammatory agents (for example a corticosterold or an NSAID), anticholinergic agents (for example, an M₁, M₂, M₁/M₂ or M₃ receptor antagonist), other 2-adrenoreceptor agonists, antiinfective agents (e.g. an antibiotic or an antiviral), and antihistamines. All suitable combinations are envisaged.

Suitable anti-inflammatory agents include corticosteroids and NSAIDs. Suitable corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothiolc acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothloic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, beta-2 integrin antagonists and adenosine receptor agonists or antagonists (e.g. adenosine 2a agonists), cytokine antagonists (e.g. chemokine antagonists) or inhibitors of cytokine synthesis. Suitable other β₂-adrenoreceptor agonists include salmeterol (e.g. as the xinafoate), salbutamol (e.g. as the sulphate or the free base), formoterol (e.g. as the fumarate), fenoterol or terbutaline and salts thereof.

Of particular interest is use of the compound of formula (I) in combination with a phosphodiesterase 4 (PDE4) inhibitor or a mixed PDE3/PDE4 inhibitor. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family as well as PDE4. Generally it is preferred to use a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity. For the purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

A method for determining IC₅₀s ratios is set out in U.S. Pat. No. 5,998,428 which is incorporated herein in full by reference as though set out herein. See also PCT application WO 00/51599 for an another description of said assay.

Suitable PDE4 inhibitors include those compounds which have a salutary therapeutic ratio, I.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1 nM of [³H]R-rolipram to a form of PDE4 which binds rolipram with a high affinity over the IC₅₀ value for inhibiting the PDE4 catalytic activity of a form which binds rolipram with a low affinity using 1 μM[³H]-cAMP as the substrate.

Most suitable are those PDE4 inhibitors which have an IC₅₀ ratio of greater than 0.5, and particularly those compounds having a ratio of greater than 1.0. Preferred compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]; these are examples of compounds which bind preferentially to the low affinity binding site and which have an IC₅₀ ratio of 0.1 or greater.

Other suitable medicament compounds include: cis-4-cyano-4-[3-(cyclopentyloxy)-4-methoxyphenyl]cyclohexane-1-carboxylic acid (also known as cilomalast) disclosed in U.S. Pat. No. 5,552,438 and its salts, esters, pro-drugs or physical forms; AWD-12-281 from elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as Cl-1018(PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P 2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Suitable antcholinergic agents are those compounds that act as antagonists at the muscarinic receptor, in particular those compounds, which are antagonists of the M₁ and M₂ receptors. Exemplary compounds include the alkaloids of the belladonna plants as illustrated by the likes of atropine, scopolamine, homatropine, hyoscyamine; these compounds are normally administered as a salt, being tertiary amines.

Particularly suitable antcholinergics include ipratropium (e.g. as the bromide), sold under the name Atrovent, oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO01/04118.

Suitable antihistamines (also referred to as H₁-receptor antagonists) include any one or more of the numerous antagonists known which inhibit H₁-receptors, and are safe for human use. All are reversible, competitive inhibitors of the interaction of histamine with H₁-receptors. Examples include ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic the tertiary amine group with piperizine or piperidine. Exemplary antagonists are as follows:
Ethanolamines: carbinoxamine maleate, clemastine fumarate, diphenylhydramine hydrochloride, and dimenhydrinate.
Ethylenediamines: pyrilamine amleate, tripelennamine HCl, and tripelennamine citrate.
Alkylamines: chiropheniramine and its salts such as the maleate salt, and acrivastine.
piperazines: hydroxyzine HCl, hydroxyzine pamoate, cyclizine HCl, cyclizine lactate, meclizine HCl, and cetirizine HCl.
Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H₁ receptor antagonist which may be used in combination with a PDE4 inhibitor.

Particularly suitable antihistamines include methapyrilene and loratadine.

Co-formulation compatibility is generally determined on an experimental basis by known methods and may depend on chosen type of medicament dispenser action (e.g. DPI, MDI, LSI).

The at least three active medicament components are suitably selected from the group consisting of anti-inflammatory agents (for example a corticosteroid or an NSAID), anticholinergic agents (for example, an M₁, M₂, M₁/M₂ or M₃ receptor antagonist), other β₂-adrenoreceptor agonists, antinfective agents (e.g. an antibiotic or an antiviral), and antihistamines. All suitable combinations are envisaged.

Suitably, the co-formulation compatible components comprise a β₂-adrenoreceptor agonist and a corticosteroid; and the co-formulation incompatible component comprises a PDE-4 inhibitor, an anti-cholinergic or a mixture thereof. The P2-adrenoreceptor agonists may for example be salbutamol (e.g., as the free base or the sulphate salt) or salmeterol (e.g., as the xinafoate salt) or formoterol (eg as the fumarate salt). The corticosteroid may for example, be a beclomethasone ester (e.g., the dipropionate) or a fluticasone ester (e.g., the propionate) or budesonide.

In one example, the co-formulation compatible components comprise fluticasone propionate and salmeterol, or a salt thereof (particularly the xinafoate salt) and the co-formulation incompatible component comprises a PDE-4 inhibitor, an anti-cholinergic (e.g. ipratropium bromide or tiotropium bromide) or a mixture thereof.

In another example, the co-formulation compatible components comprise budesonide and formoterol (e.g. as the fumarate salt) and the co-formulation incompatible component comprises a PDE-4 inhibitor, an anti-cholinergic (e.g. ipratropium bromide or tiotropium bromide) or a mixture thereof.

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

Aerosol formulations suitable for use with metered dose inhaler (MDI) dispensers typically comprise a propellant. Suitable propellants include P11, P114 and P12, and the CFC-free hydrofluoroalkane propellants HFA-134a and HFA-227.

The MDI aerosol formulation may additionally contain a volatile adjuvant such as a saturated hydrocarbon for example propane, n-butane, isobutane, pentane and Isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

A polar co-solvent such as C₂₋₆ aliphatic alcohols and polyols e.g. ethanol, isopropanol and propylene glycol, preferably ethanol, may be included in the MDI aerosol formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise the part or all of the medicament component, such formulations being commonly referred to as solution formulations.

A surfactant may also be employed in the MDI aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

The device of the invention is in one aspect suitable for dispensing medicament for the treatment of respiratory disorders such as disorders of the lungs and bronchial tracts including asthma and chronic obstructive pulmonary disorder (COPD). In another aspect, the invention is suitable for dispensing medicament for the treatment of a condition requiring treatment by the systemic circulation of medicament, for example migraine, diabetes, pain relief e.g. inhaled morphine.

Accordingly, there is provided the use of a device according to the invention for the treatment of a respiratory disorder, such as asthma and COPD. Alternatively, the present invention provides a method of treating a respiratory disorder such as, for example, asthma and COPD, which comprises administration by inhalation of an effective amount of medicament product as herein described from a device of the present invention.

The amount of any particular medicament compound or a pharmaceutically acceptable salt, solvate or physiologically functional derivative thereof which is required to achieve a therapeutic effect will, of course, vary with the particular compound, the route of administration, the subject under treatment, and the particular disorder or disease being treated. The medicaments for treatment of respiratory disorders herein may for example, be administered by inhalation at a dose of from 0.0005 mg to 10 mg, preferably 0.005 mg to 0.5 mg. The dose range for adult humans is generally from 0.0005 mg to 100 mg per day and preferably 0.01 mg to 1 mg per day.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A medicament dispenser device for use in the delivery of a multi-component combination medicament product, the device comprising
a first medicament container containing plural co-formulation compatible medicament components;
first release means for releasing the contents of the first medicament container for delivery thereof;
at least one further medicament container, each containing at least one co-formulation incompatible medicament component; and
at least one further release means for releasing the contents of each at least one further medicament container for delivery thereof,
wherein the at least one co-formulation incompatible medicament component is kept separate from the plural co-formulation compatible components until the point of release thereof for delivery in combination.

2. A medicament dispenser device according to claim 1, wherein the dispenser device comprises a first medicament container containing two co-formulation compatible medicament components and one further medicament container containing one co-formulation incompatible medicament component.

3. A medicament dispenser device according to claim 1, wherein the dispenser device comprises a first medicament container containing two co-formulation compatible medicament components and one further medicament container containing two co-formulation incompatible medicament components.

4. A medicament dispenser device according to claim 1, wherein the dispenser device comprises a first medicament container containing two co-formulation compatible medicament components a second medicament container containing one co-formulation incompatible medicament component and a third medicament container containing one co-formulation incompatible medicament component.

5. A medicament dispenser device according to claim 1, wherein the first medicament container and the at least one further medicament container are suitable for use with a medicament dispenser selected from the group consisting of a reservoir dry powder inhaler (RDPI), a multi-dose dry powder inhaler (MDPI), a unit dose dry powder inhaler (UDPI), a metered dose inhaler (MDI) and a liquid spray inhaler (LSI).

6. A medicament dispenser device according to claim 1, wherein the first and at least one further medicament container are of a similar type.

7. A medicament dispenser device according to claim 1, wherein the first and at least one further medicament container are of a different type.

8. A medicament dispenser device according to claim 1, wherein the first release means and the at least one further release means are coupled together

9. A medicament dispenser device according to claim 1, wherein the device further comprises a mixing chamber including a first inlet for receiving the released contents of the first medicament container; a second inlet for receiving the released contents of the at least one further medicament container; and an outlet for delivery of combination medicament product therefrom.

10. A medicament dispenser device according to claim 1, wherein the plural co-formulation compatible medicament components and the at least one co-formulation incompatible medicament component are selected from the group consisting of anti-inflammatory agents, anticholinergic agents, other β₂-adrenoreceptor agonists, anti-infective agents and antihistamines.

11. A medicament dispenser device according to claim 10, wherein said anti-inflammatory agents are selected from the group consisting of corticosteroids and NSAIDs.

12. A medicament dispenser device according to claim 11, wherein said corticosteroids are selected from the group consisting of methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters, budesonide, flunisolide, mometasone esters, triamcinolone acetonide, rofleponide, ciclesonide, butixocort propionate, RPR-106541 and ST-126.

13. A medicament dispenser device according to claim 12, wherein said corticosteroids are selected from the group consisting of fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

14. A medicament dispenser device according to claim 11, wherein said NSAIDs are selected from the group consisting of sodium cromoglycate, nedocromil sodium, phosphodiesterase (PDE) inhibitors, leukotriene antagonists, inhibitors of leukotriene synthesis, iNOS inhibitors, tryptase and elastase inhibitors, beta-2 integrin antagonists, adenosine receptor agonists or antagonists, cytokine antagonists and inhibitors of cytokine synthesis.

15. A medicament dispenser device according to claim 14, wherein said phosphodiesterase (PDE) inhibitors are selected from the group consisting of theophylline, PDE4 inhibitors and mixed PDE3/PDE4 inhibitors.

16. A medicament dispenser device according to claim 10, wherein said other β₂-adrenoreceptor agonists are selected from the group consisting of salmeterol, salbutamol, formoterol, fenoterol, terbutaline and any salts or solvates thereof.

17. A medicament dispenser device according to claim 10, wherein said anticholinergic agents are selected from the group consisting of M₁, M₂, M₁/M₂ and M₃ receptor antagonists.

18. A medicament dispenser device according to claim 17, wherein said anticholinergic agents are selected from the group consisting of ipratropium, oxitropium, tiotropium and any salts or solvates thereof.

19. A medicament dispenser device according to claim 1, wherein the plural co-formulation compatible medicament components comprise a β₂-adrenoreceptor agonist and a corticosteroid; and the at least one further co-formulation incompatible medicament component is selected from the group consisting of a PDE-4 inhibitor, an anti-cholinergic, and any mixtures thereof.

20. A medicament dispenser device according to claim 19, wherein the β₂-adrenoreceptor agonists is selected from the group consisting of salbutamol, salmeterol, formoterol and any salts and solvates thereof, and the corticosteroid is selected from the group consisting of beclomethasone ester, a fluticasone ester, budesonide and any salts of solvates thereof.

21. A medicament dispenser device according to claim 20, wherein the plural co-formulation compatible medicament components comprise fluticasone propionate and salmeterol xinafoate; and the co-formulation incompatible component is selected from the group consisting of a PDE-4 inhibitor, an anti-cholinergic, and any mixtures thereof.

22. A medicament dispenser device according to claim 20, wherein the plural co-formulation compatible medicament components comprise budesonide and formoterol fumarate; and the co-formulation incompatible component is selected from the group consisting of a PDE-4 inhibitor, an anti-cholinergic, and any mixtures thereof.

23. A medicament pack for use with the medicament dispenser device according to claim 1, the pack comprising
a first medicament container containing plural co-formulation compatible medicament components; and
at least one further medicament container, each at least one further medicament container containing at least one co-formulation incompatible medicament component; and
wherein the at least one co-formulation incompatible medicament component is kept separate from the plural co-formulation compatible components.

24. A medicament pack according to claim 23, wherein the plural co-formulation compatible medicament components and the at least one co-formulation incompatible medicament component are selected from the group consisting of anti-inflammatory agents, anticholinergic agents, other β₂-adrenoreceptor agonists, anti-infective agents and antihistamines.

25. A medicament pack according to claim 24, wherein said anti-inflammatory agents are selected from the group consisting of corticosteroids and NSAIDs.

26. A medicament pack according to claim 25, wherein said corticosteroids are selected from the group consisting of methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters, budesonide, flunisolide, mometasone esters, triamcinolone acetonide, rofleponide, ciclesonide, butixocort propionate, RPR-106541 and ST-126.

27. A medicament pack according to claim 26, wherein said corticosteroids are selected from the group consisting of fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11-hydroxy-16α-methyl-3-oxo-androsta-1,4-diene-17β-carbothioic acid S-fluoromethyl ester.

28. A medicament pack according to claim 25, wherein said NSAIDs are selected from the group consisting of sodium cromoglycate, nedocromil sodium, phosphodiesterase (PDE) inhibitors, leukotriene antagonists, inhibitors of leukotriene synthesis, iNOS inhibitors, tryptase and elastase inhibitors, beta-2 integrin antagonists, adenosine receptor agonists or antagonists, cytokine antagonists and inhibitors of cytokine synthesis.

29. A medicament pack according to claim 28, wherein said phosphodiesterase (PDE) inhibitors are selected from the group consisting of theophylline, PDE4 inhibitors and mixed PDE3/PDE4 inhibitors.

30. A medicament pack according to claim 24, wherein said other β₂-adrenoreceptor agonists are selected from the group consisting of salmeterol, salbutamol, formoterol, fenoterol, terbutaline and any salts or solvates thereof.

31. A medicament pack according to claim 24, wherein said anticholinergic agents are selected from the group consisting of M₁, M₂, M₁/M₂ and M₃ receptor antagonists.

32. A medicament pack according to claim 31, wherein said anticholinergic agents are selected from the group consisting of ipratropium, oxitropium, tiotropium and any salts or solvates thereof.

33. A medicament pack according to claim 23, wherein the plural co-formulation compatible medicament components comprise a β₂-adrenoreceptor agonist and a corticosteroid; and the at least one further co-formulation incompatible medicament component is selected from the group consisting of a PDE-4 inhibitor, an anti-cholinergic, and any mixtures thereof.

34. A medicament dispenser device according to claim 33, wherein the β₂-adrenoreceptor agonist is selected from the group consisting of salbutamol, salmeterol, formoterol and any salts and solvates thereof; and the corticosteroid is selected from the group consisting of beclomethasone ester, a fluticasone ester, budesonide and any salts of solvates thereof.

35. A medicament dispenser device according to claim 34, wherein the plural co-formulation compatible medicament components comprise fluticasone propionate and salmeterol xinafoate; and the co-formulation incompatible component is selected from the group consisting of a PDE-4 inhibitor, an anti-cholinergic, and any mixtures thereof.

36. A medicament dispenser device according to claim 34, wherein the plural co-formulation compatible medicament components comprise budesonide and formoterol fumarate; and the co-formulation incompatible component is selected from the group consisting of a PDE-4 inhibitor, an anti-cholinergic, and any mixtures thereof.

37. Refill medicament pack in kit of parts form for use with the medicament dispenser device according to claim 1, the refill pack comprising
as a first part of said kit, a first medicament container containing plural co-formulation compatible medicament components; and
as a further part of the kit, at least one further medicament container, each at least one further medicament container containing at least one co-formulation incompatible medicament component; and
wherein the at least one co-formulation incompatible medicament component is kept separate from the plural co-formulation compatible components.
